SEQUENCE LISTING (4 sequences)

SEQ ID NO: 1 - protein, 10 aa, artificial sequence
  synthetic peptide
ELAGIGILTV

SEQ ID NO: 2 - protein, 9 aa, artificial sequence
  synthetic peptide
IMDQVPFSV

SEQ ID NO: 3 - DNA, 29 bp, artificial sequence
  synthetic oligonucleotide
caccatcaccatcaccattgagcggccgc

SEQ ID NO: 4 - DNA, 13 bp, artificial sequence
  synthetic oligonucleotide
gctagccaccggt

(19) United States
(12) Patent Application Publication
(10) Pub. No.: US 2012/0315269 A1
(43) Pub. Date: Dec. 13, 2012

(54) IMMUNOGLOBULIN-LIKE TRANSCRIPT (ILT) RECEPTORS AS CD8 ANTAGONISTS

(75) Inventors: Eynav Klechevsky, Haifa (IL); Jacques F. Banchereau, Montclair, NJ (US); Gerard Zurawski, Midlothian, TX (US); Sandra Zurawski, Midlothian, TX (US)

(73) Assignee: BAYLOR RESEARCH INSTITUTE, Dallas, TX (US)

(21) Appl. No.: 13/465,371

(22) Filed: May 7, 2012

Related U.S. Application Data
(60) Provisional application No. 61/482,859, filed on May 5, 2011.

(51) Int. Cl. A61K 39/395; A61P 11/06; A61P 37/00; A61P 37/06; A61P 31/14; A61K 35/12; A61P 17/00

(52) U.S. Cl. 424/133.1; 424/178.1; 424/130.1; 424/93.7

(57) ABSTRACT

Compositions and methods for inhibiting effector CD8⁺ T cell priming by Langerhans cells (LCs), together with promotion of the production of IL-4 and IL-10 are disclosed herein. The findings of the present indicate that immunoglobulin-like transcript (ILT) inhibitory receptors expressed on dermal CD14+ DCs represent natural counterparts of the anti-CD8 mAbs. Accordingly, blocking ILT2 or ILT4 on dermal CD14⁺ DCs enhanced the generation of effector polyfunctional CD8⁺ T cells. Conversely, soluble ILT2 and ILT4 act as CD8-antagonists that inhibit effector CD8⁺ T cell priming by LCs, together with promoting the production of IL-4 and IL-10. The results presented herein indicate that ILT receptor family members can skew the polarization of CD8⁺ T cell responses and strategies to block ILT expression on dendritic cells (DCs) may be useful to augment dendritic cell function to enhance responses to cancer or chronic viral infections.

### CROSS-REFERENCE TO RELATED APPLICATIONS

This application claims priority to U.S. Provisional Application Ser. No. 61/482,859, filed May 5, 2011, the entire contents of which are incorporated herein by reference.

### STATEMENT OF FEDERALLY FUNDED RESEARCH

This invention was made with U.S. Government support under Contract Nos. RO-1 CA78846, RO-1 CA85540, PO-1 CA84512, and U-19 AI-57234 awarded by the National Institutes of Health (NIH). The government has certain rights in this invention.

### TECHNICAL FIELD OF THE INVENTION

The present invention relates in general to immunity and tolerance induction, and more particularly, to soluble immunoglobulin-like transcripts ILT2 and ILT4 that act as CD8-antagonists that inhibit effector CD8⁺ T cell priming by Langerhans cells (LCs), together with promoting the production of IL-4 and IL-10.

### REFERENCE TO A SEQUENCE LISTING

The present application includes a Sequence Listing, which has been submitted in ASCII format via EFS-Web and is hereby incorporated by reference in its entirety. Said ASCII copy, created on May 1, 2012, is named BHCS1129SL.txt and is 1,174 bytes in size.

### BACKGROUND OF THE INVENTION

Without limiting the scope of the invention, its background is described in connection with strategies to augment dendritic cell function to enhance responses to cancer or chronic viral infections.

U.S. Patent Publication No. 20100135997 (Jakobsen et al. 2010) provides monomeric and dimeric polypeptide fusions comprising mutated human ILT molecules and immunoglobulin Fc segments. Such compositions are said to be useful, either alone or associated with a therapeutic agent, for targeting cells expressing Class I pMHC molecules.

U.S. Patent Publication No. 20110034675, filed by Ponath et al., is directed to ILT3 binding molecules and uses therefor. Briefly, the invention is said to provide binding molecules that specifically bind to ILT3, e.g., human ILT3 (hILT3), on antigen presenting cells, such as for example, monocytes, macrophages and dendritic cells (DC), e.g., monocyte-derived dendritic cells (MDDC). The molecules bind to hILT3 with high affinity and downmodulate immune responses in vitro, e.g., downmodulating alloimmune responses; the production of inflammatory cytokines by dendritic cells, e.g., monocyte-derived dendritic cells (MDDC); the upregulation of costimulatory molecules by DC, e.g., MDDC; and/or calcium flux in monocytes. In addition, the binding molecules are said to upregulate the expression of inhibitory receptors on dendritic cells, e.g., immature dendritic cells. Finally, these same binding molecules that downmodulate immune responses in vitro, are said to be immunostimulatory in vivo.

U.S. Pat. No. 6,180,600 issued to Jameson et al. (2001) discloses compounds that inhibit CD8 mediated T cell activation and that have a molecular surface that corresponds to the molecular surface of human CD8 at amino acids 38-46 and/or 53-56 and/or 60-67 and pharmaceutical compositions comprising such compounds are disclosed. The Jameson invention further discloses methods of inhibiting activation of a human T cell. The methods comprise contacting a T cell with a compound that inhibits CD8 mediated T cell activation and that has a molecular surface that corresponds to the molecular surface of human CD8 at amino acids 38-46 and/or 53-56 and/or 60-67. Methods of treating an individual suspected of suffering from or susceptible to graft versus host disease and/or organ rejection are disclosed. The methods comprise administering an effective amount of a compound that inhibits CD8 mediated T cell activation and that has a molecular surface that corresponds to the molecular surface of human CD8 at amino acids 34-46 and/or 53-56 and/or 60-67.

### SUMMARY OF THE INVENTION

The present invention describes compositions and methods to block immunoglobulin-like transcript (ILT) expression on dendritic cells (DCs) to augment dendritic cell function to enhance responses to cancer or chronic viral infections. The present invention suggests blocking ILT2 or ILT4 on dermal CD14+ DCs to enhance the generation of effector polyfunctional CD8+ T cells. Conversely, soluble ILT2 and ILT4 act as CD8-antagonists that inhibit effector CD8+ T cell priming by LCs, together with promoting the production of IL-4 and IL-10.

The instant invention in one embodiment provides An immunostimulatory composition comprising: one or more antigenic peptides, wherein the antigenic peptides are representative of one or more epitopes of the one or more antigens implicated or involved in a disease or a condition against which the immune response, the prophylaxis, the therapy, or any combination thereof is desired and at least one immunoglobulin-like transcript (ILT) receptor antagonist, wherein the ILT receptor is selected from the group consisting of ILT2, ILT4, ILT5, or any combinations thereof obtained from one or more dermal CD14⁺ dendritic cells (DCs). In one aspect of the composition described hereinabove the one or more antigenic peptides is further defined as a conjugate, wherein the conjugate comprises the antigenic peptide loaded, recombinantly linked or coupled chemically or with a recombinant linker to a dendritic cell (DC)-specific antibody or fragment thereof. In another aspect the DC-specific antibody or fragment thereof is selected from an antibody that specifically binds to MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, and ASGPR. In a specific aspect the ILT antagonist and/or the DC-specific antibody are humanized.

In a related aspect the antigenic peptides comprise at least one of a peptide or protein selected from gag, pol, env, Nef protein, reverse transcriptase, PSA-tetramer, a HIVgag-derived p24-PLA HIV gag p24 (gag), and other HIV components, hepatitis viral antigens, influenza viral antigens and peptides selected from the group consisting of hemagglutinin, neuraminidase, Influenza A Hemagglutinin HA-1 from a H1N1 Flu strain, HLA-A201-FluMP (58-66) peptide tetramer, and Avian Flu (HA5-1), measles viral antigens, rubella viral antigens, rotaviral antigens, cytomegaloviral antigens, respiratory syncytial viral antigens, herpes simplex viral antigens, varicella zoster viral antigens, Japanese encephalitis viral antigens, rabies viral antigens or combinations and modifications thereof. In another aspect the antigenic peptides are cancer peptides selected from tumor associated antigens comprising antigens from leukemias and lymphomas, neurological tumors such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer, penile cancer, bone tumors, vascular tumors, cancer of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma, and leukemia. In yet another aspect the antigenic peptides are selected from at least one of CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67.

In one aspect the ILT receptor antagonist comprises a mixture of an ILT2 receptor antagonist and an ILT4 receptor antagonist, wherein a ratio of the ILT2 receptor antagonist to the ILT4 receptor antagonist is 5:95, 10:90, 20:80, 25:75, 30:70, 40:60, 50:50, 60:40, 70:30, 75:25, 80:20, 90:10, and 5:95. In another aspect the composition is adapted for subcutaneous administration, intradermal administration, or both. In another aspect the immunostimulatory composition produced or increased the production of multifunctional CD8⁺ T cells by the one or more dermal CD14⁺ DCs. In yet another aspect the multifunctional CD8⁺ T cells show increased production of one or more cytokines, wherein the cytokines comprise IFN-γ, TNF-α, IL-2, and any combinations thereof. The immunostimulatory composition described hereinabove is used for prophylaxis, therapy, or any combination thereof against cancer, HIV, chronic viral infection, or any combinations thereof. In one aspect the ILT receptor antagonist is a small molecule receptor antagonist, a soluble protein, a fusion protein, an antibody or a fragment thereof, a polypeptide, or any combinations thereof.

Another embodiment of the instant invention provides a vaccine comprising one or more antigenic peptides and at least one antagonist that inhibits the binding of immunoglobulin-like transcript (ILT) receptor to CD8, wherein the vaccine is adapted for delivery to dermal CD14⁺ dendritic cells (DCs), wherein the antigenic peptides and the antagonist are provided in an amount effective to produce an immune response, a prophylaxis, a therapy or any combination thereof in a human or an animal subject. In one aspect the one or more antigenic peptides is further defined as a conjugate, wherein the conjugate comprises the antigenic peptide loaded, recombinantly linked or coupled chemically or with a recombinant linker to a dendritic cell (DC)-specific antibody or fragment thereof. In another aspect the vaccine composition further comprises one or more optional pharmaceutically acceptable carriers and adjuvants. In a specific aspect the antagonist is an ILT2, an ILT4, or an ILT5 antagonist, and even more specifically they are humanized.

In yet another aspect the DC-specific antibody or fragment thereof is humanized and is selected from an antibody that specifically binds to MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASGPR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, and ASPGR.

In one aspect the antigenic peptides comprise at least one of a peptide or protein selected from gag, pol, env, Nef protein, reverse transcriptase, PSA-tetramer, a HIVgag-derived p24-PLA HIV gag p24 (gag), and other HIV components, hepatitis viral antigens, influenza viral antigens and peptides selected from the group consisting of hemagglutinin, neuraminidase, Influenza A Hemagglutinin HA-1 from a H1N1 Flu strain, HLA-A201-FluMP (58-66) peptide tetramer, and Avian Flu (HA5-1), measles viral antigens, rubella viral antigens, rotaviral antigens, cytomegaloviral antigens, respiratory syncytial viral antigens, herpes simplex viral antigens, varicella zoster viral antigens, Japanese encephalitis viral antigens, rabies viral antigens or combinations and modifications thereof. In another aspect the antigenic peptides are cancer peptides selected from tumor associated antigens comprising antigens from leukemias and lymphomas, neurological tumors such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer, penile cancer, bone tumors, vascular tumors, cancer of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma and leukemia. In yet another aspect the antigenic peptides are selected from at least one of CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67.

In one aspect the ILT receptor antagonist comprises a mixture of an ILT2 receptor antagonist and an ILT4 receptor antagonist, wherein a ratio of the ILT2 receptor antagonist to the ILT4 receptor antagonist is 5:95, 10:90, 20:80, 25:75, 30:70, 40:60, 50:50, 60:40, 70:30, 75:25, 80:20, 90:10, and 5:95. In another aspect the vaccine is adapted for subcutaneous administration, intradermal administration, or both. In yet another aspect the ILT receptor antagonist is a small molecule receptor antagonist, a soluble ILT protein, a fusion protein, an antibody or a fragment thereof that binds specifically to an ILT protein, a polypeptide, or any combinations thereof.

In yet another embodiment the present invention provides a method for augmenting dendritic cell (DC) function, enhancing generation of polyfunctional CD8⁺ T cells by one or more dermal CD14⁺ dendritic cells (DCs), inducing generation of one or more cytotoxic T cells, or any combinations thereof in a human or animal subject comprising the steps of: i) isolating and purifying an antigen-antibody conjugate comprising one or more dendritic cell (DC)-specific antibodies or fragments thereof and one or more native or engineered antigenic peptides, ii) providing at least one immunoglobulin-like transcript (ILT) receptor antagonist, wherein the ILT receptor is expressed on dermal CD14⁺ dendritic cells (DCs), iii) combining the antigen-antibody conjugate with the ILT receptor antagonist to form an immunostimulatory composition, and iv) introducing the composition into the human or animal subject to augment DC function, enhance generation of polyfunctional CD8⁺ T cells by one or more dermal CD14⁺ DCs, induce generation of one or more cytotoxic T cells, or any combinations thereof.

The present invention also discloses a method of providing immunostimulation by activation of one or more dendritic cells (DCs) to a human subject comprising the steps of: (i) identifying the human subject in need of immunostimulation for the prophylaxis, the therapy or a combination thereof against the one or more viral, bacterial, fungal, parasitic, protozoal, and parasitic diseases, and allergic disorders, (ii) isolating one or more DCs from the human subject, (iii) exposing the isolated DCs to activating amounts of a composition or a vaccine comprising: one or more antigenic peptides, wherein the antigenic peptides are representative of one or more epitopes of the one or more antigens implicated or involved in the viral, the bacterial, the fungal, the parasitic, the protozoal, and the parasitic diseases, and the allergic disorders against which the immune response, the prophylaxis, the therapy, or any combination thereof is desired and at least one immunoglobulin-like transcript (ILT) receptor antagonist, wherein the ILT receptor is selected from the group consisting of ILT2, ILT4, ILT5, or any combinations thereof expressed on dermal CD14⁺ dendritic cells (DCs), and (iv) reintroducing the activated DC complex into the human subject.

In one aspect of the method disclosed hereinabove the human subject is defined further as being a participant in a pre-clinical or a clinical trial. In another aspect the antigenic peptide comprises bacterial antigens selected from pertussis toxin, filamentous hemagglutinin, pertactin, FIM2, FIM3, adenylate cyclase and other pertussis bacterial antigen components, diptheria bacterial antigens, diptheria toxin or toxoid, other diptheria bacterial antigen components, tetanus bacterial antigens, tetanus toxin or toxoid, other tetanus bacterial antigen components, streptococcal bacterial antigens, gram-negative bacilli bacterial antigens, Mycobacterium tuberculosis bacterial antigens, mycolic acid, heat shock protein 65 (HSP65), Helicobacter pylori bacterial antigen components; pneumococcal bacterial antigens, haemophilus influenza bacterial antigens, anthrax bacterial antigens, and rickettsiae bacterial antigens. In yet another aspect the antigenic peptide comprises fungal antigens selected from candida fungal antigen components, histoplasma fungal antigens, cryptococcal fungal antigens, coccidiodes fungal antigens and tinea fungal antigens.

In another aspect the antigenic peptide comprises protozoal and parasitic antigens antigens selected from plasmodium falciparum antigens, sporozoite surface antigens, circumsporozoite antigens, gametocyte/gamete surface antigens, blood-stage antigen pf 155/RESA, toxoplasma, schistosomae antigens, leishmania major and other leishmaniae antigens and trypanosoma cruzi antigens. In yet another aspect the antigenic peptide comprises antigens involved in autoimmune diseases, allergy, and graft rejection selected from diabetes, diabetes mellitus, arthritis, multiple sclerosis, myasthenia gravis, systemic lupus erythematosis, autoimmune thyroiditis, dermatitis, psoriasis, Sjogren's Syndrome, alopecia greata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis. In one aspect the antigenic peptide comprises antigens involved in allergic disorders selected from Japanese cedar pollen antigens, ragweed pollen antigens, rye grass pollen antigens, animal derived antigens, dust mite antigens, feline antigens, histocompatibility antigens, and penicillin and other therapeutic drugs.

Another embodiment of the present invention describes a composition for modulating an immune response, suppressing an immune response, or both for a prophylaxis, a therapy or any combination thereof in a human or animal subject comprising: i) one or more an anti-dendritic cell (DC)-specific antibodies, wherein the anti-DC-specific antibody may be a conjugate, wherein the conjugate comprises the one or more anti-DC-specific antibodies or fragments thereof loaded or chemically coupled with one or more antigenic peptides, wherein the antigenic peptides are representative of one or more epitopes of the one or more antigens implicated or involved in a disease or a condition against which the modulation or the suppression of the immune response for the prophylaxis, the therapy or any combination thereof is desired, ii) one or more immunoglobulin-like transcript (ILT) receptors, receptor agonist, receptor-like segments, or fragments thereof selected from the group consisting of ILT2, ILT4, ILT5, or any combinations thereof, wherein the ILT receptor is in a form of a fusion protein, a monomeric, dimeric or multimeric polypeptide complex, an antibody, or any combinations thereof, and iii) a pharmaceutically acceptable carrier, wherein the antibodies and the ILT receptors are each comprised in an amount such that, in combination with the other, are effective to modulate or suppress the immune response, for prophylaxis, for therapy or any combination thereof in the human or animal subject in need thereof. The composition as disclosed hereinabove is used for the prophylaxis, the therapy, or both of one or more diseases or conditions selected from the group consisting of asthma, eczema, allograft rejection, graft-versus-host disease, hepatitis, and autoimmune disorders.

In yet another embodiment the present invention relates to a method for suppressing dendritic cell (DC) function, decreasing generation of polyfunctional CD8⁺ T cells by one or more dermal CD14⁺ dendritic cells (DCs), one or more cytotoxic T cells, or both, stimulating generation of one or more Type 2 cytokine-secreting CD8⁺ T cells (TC2), or any combinations thereof in a human or animal subject comprising the steps of: (i) isolating and purifying one or more dendritic cell (DC)-specific antibodies or a fragment thereof, (ii) optionally loading or chemically coupling one or more native or engineered antigenic peptides to the DC-specific antibody to form an antibody-antigen conjugate, (iii) providing one or more immunoglobulin-like transcript (ILT) receptors, receptor agonist, receptor-like segments, or fragments thereof selected from the group consisting of ILT2, ILT4, ILT5, or any combinations thereof obtained from one or more dermal CD14⁺ dendritic cells (DCs), wherein the ILT receptor is in a form of a fusion protein, a monomeric, dimeric or multimeric polypeptide complex, an antibody, or any combinations thereof, (iv) contacting the antigen-antibody conjugate with the ILT receptor to form an immunosuppressive composition, and (v) introducing the composition into the human or animal subject to suppress dendritic cell (DC) function, decrease generation of polyfunctional CD8⁺ T cells by one or more dermal CD14⁺ dendritic cells (DCs), one or more cytotoxic T cells, or both, stimulate generation of one or more Type 2 cytokine-secreting CD8⁺ T cells (TC2), or any combinations thereof.

### BRIEF DESCRIPTION OF THE DRAWINGS

For a more complete understanding of the features and advantages of the present invention, reference is now made to the detailed description of the invention along with the accompanying figures and in which:

FIGS. 1A-1F show that Dermal CD14⁺ DCs prime CD8^{low} Type 2 cytokine-producing T cells (TC2): (FIG. 1A) CFSE-labeled naïve CD8⁺ T cells were primed for 7 days by skin isolated DC subsets; LCs (left), and dermal CD14⁺ DCs (right) and analyzed by flow cytometry after 48 hours expansion with anti-CD3, anti-CD28 mAbs and IL-2. Plots show the level of CD8 surface expression by the proliferated cells, (FIG. 1B) In vitro HLA-A201+ DC subsets were loaded with MART-1 peptide were used to prime naïve CD8+ T cells. Cells were analyzed following two consecutive stimulations by flow cytometry for CD8 intensity and frequency of MART-1-tetramer binding cells, (FIG. 1C) CD8 Mean Fluorescence Intensity (MFI) expression of HLA-A201-MART-1 tetramer-binding CD8 T cells, primed by in vitro peptide-loaded autologous LCs and CD14⁺ DCs. Left: Histogram shows a representative of 17 independent runs. Right: Graph shows data of 17 independent runs. Results were obtained by paired Student's t test, (FIG. 1D) CFSE-labeled naïve CD8⁺ T cells were primed for 7 days by CD40L-activated skin DC subsets; LCs, and dermal CD14⁺ DCs and further expanded The cells were then further expanded for 48 h with a combination of plate bound anti-CD3 mAb, a soluble anti-CD28 mAb and IL-2. Intracellular expression of IL-13 was assessed by flow cytometry after additional 5 h stimulation with PMA and ionomycin in the presence of monensin. Data are representative of 3 independent runs, and (FIG. 1E) CFSE-labeled naïve CD8 T cells were primed for 7 days by skin LCs and dermal CD14⁺ DCs. CFSE^{lo} cells were sorted at the end of the culture and restimulated for 24 hours with anti-CD3 and anti-CD28 mAbs. The cytokines IL-13, IL-5, IL-4, IL-10 and IFN-γ were measured in the culture supernatant by multiplex bead-based assay. FIG. 1F shows the mean fluorescence intensity of CD8 expression by Flu-MP-specific CD8⁺ T cells primed by autologous in vitro-generated peptide loaded mDC subsets; CD1a⁺ LCs and CD14⁺ DCs. Graph shows data of 8 independent runs. Results were obtained by paired Student's t test;

FIGS. 2A-2E show that Anti-CD8 mAb inhibits CD8⁺ T cells priming against allogeneic or autologous antigens: (FIG. 2A) Naïve CD8⁺ T cell proliferation, as determined by cellular [³H]thymidine incorporation, in response to allogeneic skin LCs cultured for 5 days with indicated concentration of anti-CD8 or an isotype matched control, (FIG. 2B) Plots show CFSE dilution of naïve CD8⁺ T cell (upper panel) and CD4⁺ T cell (lower panel) after 7 day culture with allogeneic in vitro and with 1 μg/ml anti-CD8 mAb or an isotype matched control, (FIG. 2C) Plots show the frequency of HLA-A201-MART-1-specific CD8⁺ T cells primed for 9 days by peptide loaded in vitro LCs in and with 1 μg/ml of anti-CD8 mAb or an isotype matched control. Data are representative of at least 5 independent runs, (FIG. 2D) Naïve CD8⁺ T cells were primed by MART-1 peptide-loaded in vitro LCs and with 5 ng/ml of anti-CD8 mAb or an isotype-matched control. Plots show the frequency and fluorescence intensity of MART-1-specific CD8+ T cells as measured by flow cytometry with a specific HLA-A201 tetramer, and (FIG. 2E) Graph shows the mean fluorescence intensity (MFI) of the MART-1 tetramer-binding CD8⁺ T cells, for each dose of anti-CD8 mAb used during primary co-culture of naïve CD8⁺ T cells and peptide-loaded HLA-A201⁺ in vitro LCs;

FIGS. 3A and 3B show that memory CD8⁺ T cell responses are CD8-independent: (FIG. 3A) Naïve CD8⁺ T cells were cultured with allogeneic in vitro LCs for 6 days in the presence of anti-CD8 mAb or isotype matched control. Graph shows Thymidine incorporation of CD8⁺ T cell 3 days after a second consecutive stimulation with autologous in vitro LCs and (FIG. 3B) Flow cytometry analysis of Flu-MP-specific HLA-A201-tetramer binding CD8⁺ T cell expansion induced by peptide-loaded autologous in vitro LCs after 9 days of culture in the presence 3 μg/ml of the anti-CD8 (RPA-T8; BD biosciences), or an isotype-matched control. Data are representative of at least 5 different runs;

FIGS. 4A-4E show that blocking CD8 leads to the generation of TC2 cells: (FIG. 4A) Naive CD8⁺ T cells were primed by allogeneic in vitro LCs and with anti-CD8 mAb or an isotype-matched control for 7 days. The CD8⁺ T cells were analyzed by flow cytometry for the expression of intracellular granzyme A, granzyme B and perforin, (FIG. 4B) CFSE-labeled naïve CD8⁺ T cells were primed with allogeneic in vitro LCs for 7 days. The CFSE^{lo} CD8⁺ T cells were sorted and restimulated for 24 hours with anti-CD3 and anti-CD28 mAbs. The production of IL-13, IL-5, IL-4, IL-10, IL-2 and IFN-γ were measured in the culture supernatant by multiplex bead-based assay. Graphs show data of 4 independent runs, (FIG. 4C) Allogeneic CFSE-labeled naïve CD8⁺ T cells were primed for 7 days by skin dendritic cell subsets; LCs or dermal CD14⁺ DCs and with anti-CD8 or an isotype matched control. The cells were then further expanded for 48 h with a combination of plate bound anti-CD3 mAb, a soluble anti-CD28 mAb and IL-2. Intracellular expression of IL-2, IL-4 and IL-13 was assessed by flow cytometry after additional 5 h stimulation with PMA and ionomycin in the presence of monensin. Plots show the production of the above cytokines by the distinct CD8⁺ T cell cultures, (FIG. 4D) Naive CD8⁺ T cells were primed by allogeneic in vitro LCs and with anti-CD8 mAb or an isotype matched control. After 7 days, the CD8⁺ T cells were analyzed by flow cytometry for the surface expression of CD30, GITR, CD40L, CD25, and 41BB, and (FIG. 4E) Skin LCs or dermal CD14⁺ DCs were cultured with CFSE-labeled naïve CD8⁺ T cells for 8 days. Anti-CD8 mAb, or an isotype-matched control was added to the cultures as indicated. Cells were assessed for intracellular expression of intracellular IFN-γ and CD40L after overnight expansion with anti-CD3 and anti-CD28 mAbs and additional 5 hours restimulation with PMA and Ionomycin in the presence of monensin. Data are representative of 2 independent runs;

FIGS. 5A-5C show the expression analysis of the ILT family receptors by the skin DC subsets: (FIG. 5A) Gene expression analysis of the ILT family receptors ILT2, ILT3, ILT4 and ILT5 by sorted skin DC subsets: LCs and dermal CD14⁺ DCs. Graphs show that raw value gene expression of 3 different individuals, (FIGS. 5B-1, 5B-2, 5B-3) Immunofluorescence staining of ILT5 on section of human dermis. ILT5 is visualized in green; CD14 in red; cell nuclei in blue. Data are representative of 2 independent runs, and (FIG. 5C) Flow cytometry analysis of the ILT family receptors on the surface of skin DC subsets: LCs and dermal CD14⁺ DCs. Data are representative of 3 independent runs;

FIG. 6 shows the flow cytometry analysis of the ILT family receptors on the surface of dermal CD14⁺ DCs that were activated for 24 hours with CD40L or a combination of CD40L and a Toll-Like Receptor (TLR) agonist; TLR2-ligand (Pam3; 50 ng/ml), TLR3-ligand (PolyI:C; 10 μg/ml) and TLR4-ligand (LPS; 50 ng/ml). Data are representative of 2 independent runs.

FIGS. 7A-7C show that soluble ILT2 and ILT4 inhibit the generation of effector CD8⁺ T cells by LCs: (FIG. 7A) CD40L-activated in vitro LCs were cultured at ratio 1:20 with allogeneic CFSE-labeled naïve CD8⁺ T cells and with the indicated Fc-fusion proteins (at 20 μg/ml). The cultures were supplemented with IL-7 and IL-2 or day 0 and day 2, respectively. After 2 consecutive stimulations, the CD8+ T cells were analyzed by flow cytometry for the dilution of CFSE and the intracellular expression of IL-4, IL-10 granzyme A and granzyme B, (FIG. 7B) CD40L-activated skin LCs were cultured at ratio 1:40 with allogeneic CFSE-labeled naïve CD8⁺ T cells and with the indicated Fc-fusion proteins (at 20 μg/ml). The cultures were supplemented with IL-7 and IL-2 on day 0 and day 2, respectively. After 9 days, the cells were restimulated with autologous DCs for 24 hours and the CD8⁺ T cells were assessed by flow cytometry for the dilution of CFSE dye and the intracellular expression of granzyme A and granzyme B, and (FIG. 7C) CD40L-activated skin LCs were cultured at ratio 1:40 with allogeneic CFSE-labeled naïve CD8⁺ T cells and with the indicated Fc-fusion proteins (at 20 μg/ml). The cultures were supplemented with IL-7 and IL-2 on day 0 and day 2, respectively. After 10 days, the cells were expanded with a combination of plate bound anti-CD3 mAb, a soluble anti-CD28 mAb and IL-2 for 24 hours. The dilution of CFSE dye and intracellular expression of IFN-γ, and TNF-α were assessed after additional 5 h stimulation with PMA and ionomycin in the presence of monensin. Graph shows the relative CFSE^{lo} populations based on their cytokine expression profile;

FIGS. 8A-8C show that anti-ILT4 enhances the generation of polyfunctional CD8⁺ T cells: (FIG. 8A) Dermal CD14⁺ DCs were cultured at ratio of 1:40 with allogeneic CFSE-labeled naïve CD8⁺ T cells and with 1:100-dilution of mouse serum vaccinated with soluble ILT-Fc molecules. After 9 days, the cells were expanded with anti-CD3, anti-CD28 mAbs and IL-2 for 48 hours and assessed following 5 hours of reactivation with PMA and Ionomycin for the dilution of CFSE dye and intracellular expression of IL-13, IL-10 and IFN-γ by flow cytometry. Skin LCs served as a control, (FIG. 8B) mAbs specific to anti-ILT2 (mouse IgG1 clone 3F1) and anti-ILT4 (Rat IgM clone 27D6) were added at 20 μg/ml to co-cultures of dermal CD 14⁺ DCs and naïve CD8⁺ T cells. Skin LCs served as a control. As in FIG. 8A, the cells were analyzed by flow cytometry for the dilution of CFSE dye and the expression of intracellular IFN-γ, TNF-α and IL-2. Graph shows the relative populations based on their cytokine expression profile, and (FIG. 8C) Dermal CD14+ DCs were cultured at ratio 1:40 with allogeneic CFSE-labeled naïve CD8⁺ T cells and 1:100-dilution of pulled serum of 3 mice vaccinated with a soluble ILT4-Fc or a control Fc fusion proteins. After 9 days the cells were assessed by flow cytometry for the expression of intracellular granzyme B. Histogram shows the relative expression by the cells that diluted CFSE dye; and

FIGS. 9A-9D is a schematic of a model showing the action of ILT2 and ILT4 as CD8-antagonists to prevent efficient CTL priming by human dermal CD14⁺ DCs: (FIG. 9A) LCs prime high avidity polyfunctional effector CD8⁺ T cells, (FIG. 9B) Blocking CD8 during priming of naïve CD8+ T cells and LCs lead to the generation of type-2 cytokine secretion T cells. Similarly in FIG. 9C ILT2 and ILT4 receptors that are expressed by dermal CD14⁺ DCs resulting in suboptimal priming of effector CD8⁺ T cells and Type 2 cytokines, by possibly competing with CD8 on its binding to MHC class I, and (FIG. 9D) Viruses and tumor cells may utilize the ILT receptors to evade immunity.

FIGS. 10A to 10C show expression analysis of the ILT family receptors by the skin DC subsets, FIG. 10A shows a flow cytometry analysis of the ILT2 and ILT4 receptors on the surface of LCs and dermal CD 14⁺ DCs (black histogram), grey histogram represents isotype control. Data are representative of 4 independent studies, FIGS. 10B-1 (CD14), 10B-2 (ILT2), 10B-3 (overlay), 10B-4 (isotype control), 10B-5 (isotype control), 10B-6 (overlay) show immunofluorescence staining of ILT2, and FIGS. 10C-1 (CD14), 10C-2 (ILT4), 10C-3 (overlay), 10C-4 (isotype control), 10C-5 (isotype control), 10C-6 (overlay) show immunofluorescence staining of ILT4 receptors on sections of human dermis. ILT is visualized in green, CD14 in red and cell nuclei in blue.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an,” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “Antigen Presenting Cells” (APC) refers to cells that are capable of activating T cells, and include, but are not limited to, certain macrophages, B cells and dendritic cells. “Dendritic cells” (DCs) refers to any member of a diverse population of morphologically similar cell types found in lymphoid or non-lymphoid tissues. These cells are characterized by their distinctive morphology, high levels of surface MHC-class II expression (Steinman, et al., Ann. Rev. Immunol. 9:271 (1991); incorporated herein by reference for its description of such cells). These cells can be isolated from a number of tissue sources, and conveniently, from peripheral blood, as described herein. Dendritic cell binding proteins refers to any protein for which receptors are expressed on a dendritic cell. Examples include GM-CSF, IL-1, TNF, IL-4, CD40L, CTLA4, CD28, and FLT-3 ligand.

For the purpose of the present invention, the term “vaccine composition” is intended to mean a composition which can be administered to humans or to animals in order to induce an immune system response; this immune system response can result in a production of antibodies or simply in the activation of certain cells, in particular antigen-presenting cells, T lymphocytes and B lymphocytes. The vaccine composition can be a composition for prophylactic purposes or for therapeutic purposes, or both. As used herein, the term “antigen” refers to any antigen, which can be used in a vaccine, whether it involves a whole microorganism or a subunit, without regard to its specific configuration: peptide, protein, glycoprotein, polysaccharide, glycolipid, lipopeptide, etc. They may be viral antigens, bacterial antigens, or the like; the term “antigen” also comprises the polynucleotides, the sequences of which are chosen so as to encode the antigens whose expression by the individuals to which the polynucleotides are administered is desired, in the case of the immunization technique referred to as DNA immunization. They may also be a set of antigens, in particular in the case of a multivalent vaccine composition which comprises antigens capable of protecting against several diseases, and which is then generally referred to as a vaccine combination, or in the case of a composition which comprises several different antigens in order to protect against a single disease, as is the case for certain vaccines against whooping cough or the flu, for example. The term “antibodies” refers to immunoglobulins, whether natural or partially or wholly produced artificially, e.g. recombinant. An antibody may be monoclonal or polyclonal. The antibody may, in some cases, be a member of one, or a combination immunoglobulin classes, including: IgG, IgM, IgA, IgD, and IgE.

Non-limiting examples of allergens or antigens that cause asthma include pollens (grass, tree and weeds), pet or insect dander, perfumes or scents, food (corn, wheat, eggs, milk, seafood, legumes, soy, tree nuts), fungi, seeds, nuts, alcohol, plant secretions, drugs (e.g., penicillin or other antibiotics, salicylates), insect bites (bees, wasps, spiders, flies, dust mites), natural and synthetic compounds (e.g., latex), animal products (fur, dander, wool), mold spores, and metal. Specific examples of compounds, peptides, carbohydrates, proteins, lipids and combinations thereof that may be attached to the antibodies and binding fragments of the present invention may be found in, e.g., the allergome database, e.g., www.allergome.org, relevant portions incorporated herein by reference.

The term “adjuvant” refers to a substance that enhances, augments or potentiates the host's immune response to a vaccine antigen.

The term “gene” is used to refer to a functional protein, polypeptide or peptide-encoding unit. As will be understood by those in the art, this functional term includes genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated.

As used in this application, the term “amino acid” means one of the naturally occurring amino carboxylic acids of which proteins are comprised. The term “polypeptide” as described herein refers to a polymer of amino acid residues joined by peptide bonds, whether produced naturally or synthetically. Polypeptides of less than about 10 amino acid residues are commonly referred to as “peptides.” A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

As used herein, the term “in vivo” refers to being inside the body. The term “in vitro” used as used in the present application is to be understood as indicating an operation carried out in a non-living system.

As used herein, the term “treatment” or “treating” means any administration of a compound of the present invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology).

As used herein, the terms “protein”, “polypeptide” or “peptide” refer to compounds comprising amino acids joined via peptide bonds and are used interchangeably. An amino acid or “polypeptide” is a polymer of amino acid residues joined by peptide bonds, whether produced naturally or synthetically. Polypeptides of less than about 10 amino acid residues are commonly referred to as “peptides.” A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

Antibodies against the proteins of the invention can be prepared by well-known methods using a purified protein according to the invention or a (synthetic) fragment derived therefrom as an antigen. Monoclonal antibodies can be prepared, for example, by the techniques as originally described in Kohler and Milstein, Nature 256 (1975), 495, and Galfre, Meth. Enzymol. 73 (1981), 3, which comprise the fusion of mouse myeloma cells to spleen cells derived from immunized mammals. The antibodies can be monoclonal antibodies, polyclonal antibodies or synthetic antibodies as well as fragments of antibodies, such as Fab, Fv or scFv fragments etc. As used herein, an antibody is said to “specifically bind” or “immunospecifically recognize” a cognate antigen if it reacts at a detectable level with the antigen, but does not react detectably with peptides containing an unrelated sequence, or a sequence of a different heme protein. Affinities of binding partners or antibodies can be readily determined using conventional techniques, for example, those described by Scatchard et al. (Ann. N.Y. Acad. Sci. USA 51:660 (1949)) or by surface plasmon resonance (BIAcore, Biosensor, Piscataway, N.J.). See, e.g., Wolff et al., Cancer Res. 53:2560-2565 (1993).

Furthermore, antibodies or fragments thereof to the aforementioned polypeptides can be obtained by using methods that are described, e.g., in Harlow and Lane “Antibodies, A Laboratory Manual”, CSH Press, Cold Spring Harbor, 1988. For example, surface plasmon resonance as employed in the BIAcore system can be used to increase the efficiency of phage antibodies that bind to an epitope of the protein of the invention (Schier, Human Antibodies Hybridomas 7 (1996), 97.varies.105; Malmborg, J. Immunol. Methods 183 (1995), 7-13). Antibodies, which bind specifically to a wildtype or a variant protein can be used for diagnosing or prognosing a related disorder, e.g., cancer.

The present invention describes compositions and methods to block immunoglobulin-like transcript (ILT) expression on DCs to augment dendritic cell (DC) function to enhance responses to cancer or chronic viral infections. The present inventors show that various DC populations in human skin display a differential ability to prime naïve CD8+ T cell responses. Compared to Langerhans cells (LCs), dermal CD14+ DCs are less efficient at priming naïve CD8+ T cells into potent cytotoxic T lymphocytes (CTLs). The present inventors demonstrate herein that dermal CD14⁺ DCs harness CD8-antagonist receptors, Immunoglobulin-Like Transcript (ILT)2 and ILT4, to modulate efficient CTL differentiation and induce the generation of Type 2 cytokine-secreting CD8⁺ T cells (TC2) cells.

DCs are potent antigen presenting cells (APCs) responsible for inducing Ag-specific immunity and tolerance (Banchereau and Steinman, 1998; Joffre et al., 2009; Steinman and Banchereau, 2007; Tacken and Figdor, 2011). Several populations of DCs take up residence in different tissues, and carry common as well as unique biological functions (Dudziak et al., 2007; Liu, 2005; Merad et al., 2008; Pulendran et al., 1999); (Maldonado-Lopez et al., 1999); (Shortman and Liu, 2002) The healthy human skin displays at least three DCs populations—Langerhans cells (LCs) in the epidermis and interstitial CD1a⁺ and CD14⁺ DCs in the dermis (Nestle et al., 1993; Zaba et al., 2007). In the steady state, non-activated skin DCs bearing self-antigens migrate into the draining lymphoid organs leading to peripheral tolerance. However, following activation, e.g., by an invading microbe, DCs undergo maturation while migrating. Once in the draining lymph node the mature DC selects and activates microbe-specific lymphocytes. The different skin DC subsets carry specialized functions. CD14⁺ DCs that reside in the dermis are particularly efficient at controlling the differentiation of naïve B cells into plasma cells, while LCs are unable to do so (Caux et al., 1997; Dubois et al., 1998; Klechevsky et al., 2008). Epidermal LCs, however, are highly efficient at priming naïve CD8⁺ T cells into potent CTLs, when compared to CD14⁺ DC, whereas both myeloid DCs (mDCs) subsets are equally efficient at inducing a secondary CD8⁺ T cell response (Klechevsky et al., 2009; Klechevsky et al., 2008; Ratzinger et al., 2004).

T lymphocytes are also composed of subsets. The CD4⁺ T cells subset Th1 and Th2 (Mosmann et al., 1986; Mosmann and Coffman, 1989) were the first to be characterized and subsequently other subsets were identified including Tregs, Th17, Tfh and Th9 (Sakaguchi et al., 2010; Steinman, 2007; Zhu et al., 2010). CD8⁺ T cell subsets have also been described based on their phenotype and cytokine production profile (Vukmanovic-Stejic et al., 2000); (Croft et al., 1994; Seder et al., 1992)—Type 1 (TC1) express IFN-γ and TNF-α; Type 2 (TC2) produce IL-4, IL-5 and IL-13. Suppressor CD8⁺ T cells produce IL-10, TGF-β and are characterized by the expression of low levels of CD8 and CD28 (Le Gros et al., 1990; Woodland and Dutton, 2003). These findings are not restricted to in vitro studies as the fine balance between TC1 to TC2 and CD8⁺ suppressor T cells populations correlates with the patient's ability to overcome tumor overgrowth or viral infections (Apte et al., 2010; Dobrzanski et al., 2006).

Studies with CD8α or CD8β gene-targeted mice revealed that CD8 plays a key role in the maturation and function of MHC class I-restricted T lymphocytes (Connolly et al., 1988; Fung-Leung et al., 1993; Fung-Leung et al., 1991; Nakayama et al., 1994; Salter et al., 1990). The immunological synapse is the specialized junction between a T lymphocyte and an APC and consists of a central cluster of T cell receptors (TCR), including associated CD8, surrounded by a ring of adhesion molecules (Grakoui et al., 1999). There, the CD8 molecule increases the affinity of the TCR-CD8 complexes for MHC-peptide complexes by about 10-fold (Garcia et al., 1996).

Inhibitory receptors included in the immunological synapse can deliver signals that induce regulatory responses. They include: i) the non-classical MHC Class I molecules such as HLA-G (Gregori et al., 2009) and ii) the ILT family receptors (named ILT1-ILT5) (Dietrich et al., 2000). ILTs are expressed on a variety of cell types, including monocytes, dendritic cells and NK cells and negatively regulate their functions (Cella et al., 1997; Colonna et al., 1997). Two members, ILT2 and ILT4, recognize a broad range of MHC class I molecules and compete with CD8 for MHC Class I binding (Endo et al., 2008; Shiroishi et al., 2003). ILT2 modulates CD8⁺ T cell activation by blocking the binding of CD8 to MHC Class I (Shiroishi et al., 2003). Similarly PIR-B, the mouse homolog to human ILT2, regulates cytotoxic T cell triggering by competing with CD8 (Endo et al., 2008). In addition, the ILT4/HLA-G interaction contributes to the generation of T regulatory type 1 (Tr1) cells by IL-10-producing DCs (Gregori et al., 2010), while ILT3 induces CD4⁺ Th anergy and the differentiation of antigen-specific CD8⁺ T suppressor cells (Chang et al., 2002; Vlad et al., 2008).

The results presented in the present invention help in the understanding of the reasons why dermal CD14⁺ DCs were less potent than epidermal DCs in priming effector CD8⁺ T cell responses. The present inventors report herein that ILT2 and ILT4, which are expressed on human dermal CD14⁺ DCs, inhibit the differentiation of naïve CD8⁺ T cells into potent cytotoxic T cells.

DC subsets: CD34⁺-derived DCs were generated in vitro from CD34⁺-HPCs isolated from the blood of healthy volunteers given G-CSF (Neupogen) to mobilize precursor cells. HPCs were cultured at 0.5×10⁶ cells/ml in Yssel's medium (Irvine Scientific, CA) supplemented with 5% autologous serum, 50 μM β-mercaptoethanol, 1% L-glutamine, 1% penicillin/streptomycin, GM-CSF (50 ng/ml; Berlex), Flt3-L (100 ng/ml; R&D), and TNF-α (10 ng/ml; R&D) for 9 days. Media and cytokines were refreshed at day 5 of culture. Subsets of DCs, CD1a⁺CD14⁻ LCs and CD1a⁻CD14⁺ DCs were then sorted, yielding a purity of 95-99%.

Epidermal LCs (skin LCs) and dermal CD 14⁺ DCs were purified from normal human skin specimens. Specimens were incubated in bacterial protease dispase type 2 for 18 h at 4° C., and then for 2 h at 37° C. Epidermal and dermal sheets were then separated, cut into small pieces (˜1-10 mm) and placed in RPMI 1640 supplemented with 10% FBS. After 2 days, the cells that migrated into the medium were collected and further enriched using a Ficoll-diatrizoate in a density of 1.077 g/dl. DCs were purified by cell sorting after staining with anti-CD1a FITC (DAKO) and anti-CD14 APC mAbs (Invitrogen). All protocols were reviewed and approved by the institutional review board.

DC subsets were evaluated for the expression of ILT receptor after Fc blocking using 10% human AB serum with the following mAbs: anti-ILT2 #HP-F1, anti-ILT3 #ZM3.8, and anti-ILT4 #42D1 (Immunotech), ILT5 #MKT5.7H5.1 (eBiosciences). Anti-ILT5 was used to evaluate expression on skin sections using immunofluorescence analysis.

DC/T cell co-cultures: For autologous primary response assessments, naïve CD8⁺ T cells (CD8⁺CCR7⁺CD45RA⁺; 1×10⁶ cells/well) were stimulated with in vitro-generated LCs or CD14⁺ DCs (5×10⁴ cells/well) that were pre-incubated for 3 h with the HLA-A201-restricted MART-1 (26-35, ELAGIGILTV) (SEQ ID NO: 1), gp100 (209-217, IMDQVPFSV) (SEQ ID NO: 2) or a control peptide (3 μM) in the presence of anti-CD8 (RPA-T8; BD Biosciences, T8; Beckman coulter, or OKT8) or isotype control antibody (1 μg/ml unless otherwise indicated). Cells were cultured for 9 days with IL-7 (10 U/ml; R&D) and CD40L (100 ng/ml; R&D). IL-2 (10 U/ml; R&D) was added at day 3. Expansion of peptide-specific CD8⁺ T cells was determined by counting the number of cells binding peptide/HLA-A201 tetramers (Beckman Coulter) at the end of the culture period, as well as the CD8 mean florescence intensity on the tetramer binding cells. For the assessment of secondary response, autologous purified CD8⁺ T cells or sorted memory CD8⁺ T cells (1×10⁵ cells/well) were cultured with LCs or CD14⁺ DCs (5×10³ cells/well), pre-loaded with 1 μM HLA-A201-restricted Flu-MP-peptide (58-66, GILGFVFTL) (SEQ ID NO: 3), and cultured with in the presence of anti-CD8 or isotype control antibody (3 μg/ml unless otherwise indicated). Cells were cultured for 9 days in Yssel's complete medium supplemented with IL-7 (10 U/ml) and CD40L (100 ng/ml). IL-2 (10 U/ml) was added at day 3. Expansion of peptide-specific CD8⁺ T cells was determined by counting the number of cells binding peptide/HLA-A201 tetramers (Beckman Coulter) at the end of the culture period. For primary allogeneic CD8⁺ T cell culture, sorted naive T cells were cultured with allogeneic sorted mDC subsets, generated in vitro from CD34⁺ HPCs or isolated from skin at a ratio of 1:40. Where indicated, anti-CD8 mAb, soluble ILT-Fc fusion proteins or anti-ILT receptors Ab were added at the indicated concentration. Cell proliferation was assessed by the level of [³H]thymidine incorporation after 5 days, or CFSE dilution (0.5 μM CFSE; Invitrogen). Expression of effector molecules granzyme A (BD Pharmingen), granzyme B (eBiosciences) and perforin (Fitzgerald) and surface molecules CD30 (BD Biosciences), GITR (eBiosciences), CD40L, CD25 and CD137 (41BB) (all from BD Biosciences), were analyzed after 7 days by flow cytometry. For intracellular cytokine analysis, day 7-primed CD8⁺ T cells were restimulated for 24-40 h with either fresh DC or a combination of immobilized anti-CD3 (BD Biosciences) and soluble anti-CD28 (2 μg/ml; eBiosciences) in fresh medium containing IL-2 (10 IU/ml). Intracellular IL-2, IFN-γ, TNF-α, IL-4, IL-10 or IL-13 (all from BD Biosciences) expression was assessed by flow cytometry after additional 5 h stimulation with PMA (25 ng/ml; Sigma) and ionomycin (1 μM; Sigma).

Cloning of ILT-Fc molecules and the generation of ILT-specific mAbs: Vectors for the expression in stably transfected CHO-S cells of ILT-cohesin and ILT-IgFc proteins were hILT2 GI:12636295 226-1602, hILT3 GI:85567629 20-1349, hILT4 GI:2660709 3-1376 with G13C and A18G changes, or hILTS GI:2665646 3-1332 residues preceded by ACC inserted into CET1019HS-puro-SceI (Millipore) in the Fse I-Nhe I interval with either GI:308035026 520-1017 preceded by GCTAGC and followed by CACCATCACCATCACCATTGAGCGGCCGC (SEQ ID NO: 4) or GI:194381819 774-1470 preceded by GCTAGCCACCGGT (SEQ ID NO: 5) and followed by GCGGCCGC in the Nhe I-BstB I interval. Procedures for transfection and cell culture are described in Li et al. (submitted) and purification of IgFc fusion proteins in (Klechevsky et al., 2010). Procedures for immunization of mice and hybridoma derivation were as described (Klechevsky et al., 2010) and were reviewed and approved by the Institute animal care and use committee.

Immunofluorescent staining of ILT5 on human skin: Human abdominal skins were obtained from healthy donors who underwent cosmetic surgeries at Baylor University Medical Center in accordance with Institutional Review Board guidelines. Cryo-sections were fixed in cold acetone, dried and blocked for non-specific fluorescence with Fc Receptor Block and Background Buster (Innovex, CA) and goat serum. Sections were incubated in mouse anti-CD14 (clone M5E2; 10 μg/ml) and rat anti-ILT5 (clone MKT5.1; 10 μg/ml) or isotype antibodies. After washing, sections were stained with goat anti-mouse AF568 (Invitrogen) and goat anti-rat AF488 (Invitrogen), and then subsequently stained with DAPI (Molecular Probes). Single channel images were acquired using the same exposures for antibody and isotype staining and identical scaling was applied. Single channels were then assigned color and overlayed. The digital images were taken using an Olympus BX51 with a Planapo20x/0.7 or Planapo40x/0.95 objective, a Roper Coolsnap HQ camera and Metamorph software vs 6.2r6 (Molecular Devices, CA).

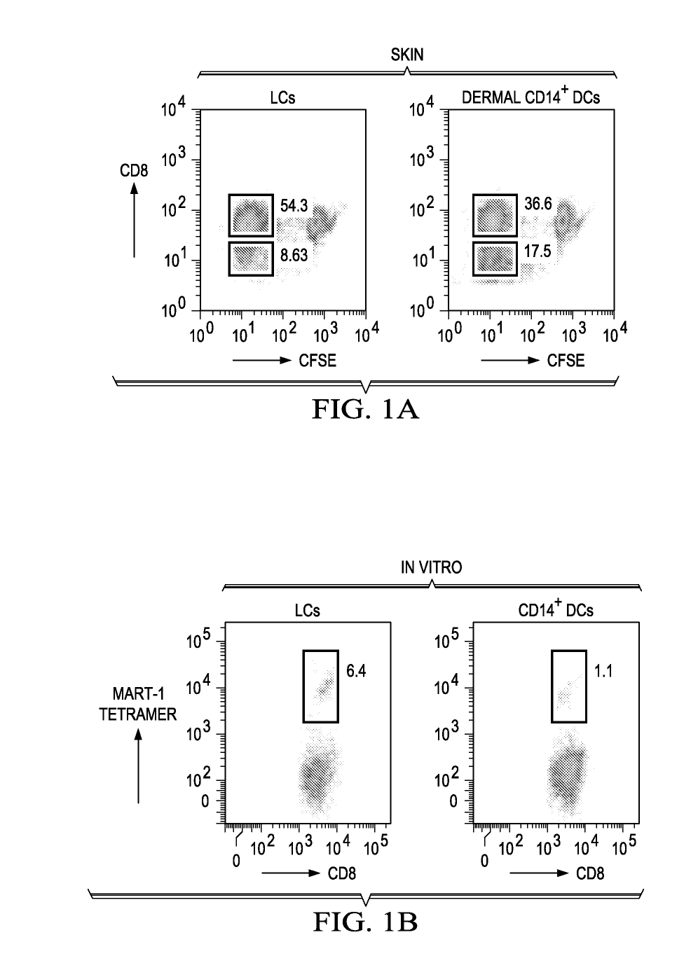

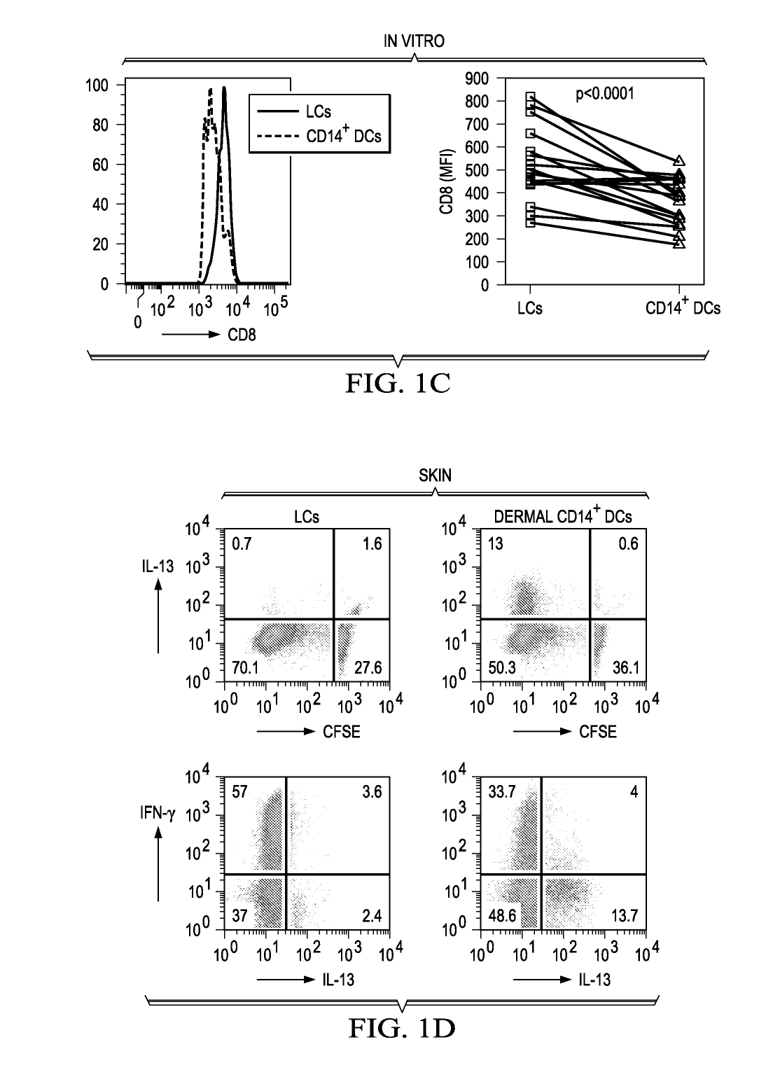

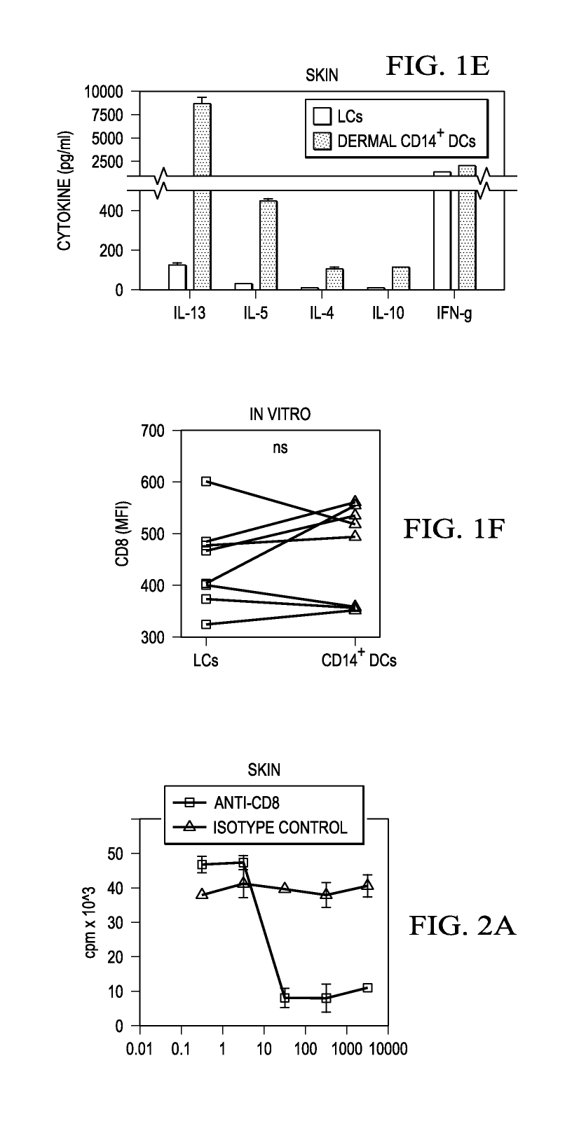

CD14⁺ DCs prime CD8^{lo} Type 2 cytokine-producing T cells (TC2): Primary human skin LCs and dermal CD14⁺ DCs were isolated from epidermal and dermal sheets, and cultured with naïve allogeneic CFSE-labeled CD8⁺ T cells. As shown in FIG. 1A, a fraction (17.5%) of the naïve CD8⁺ T cells that proliferated in response to dermal CD14⁺ DCs showed low levels of surface CD8 expression (right panel), while virtually all the CD8⁺ T cells exposed to skin LCs expressed high level of CD8 (left panels). To analyze peptide-specific-CD8⁺ T cell priming, LCs were generated by culturing HLA-A201⁺CD34⁺ HPCs (Hematopoietic Progenitor Cells) in the presence of GM-CSF, Flt3-L and TNF-α for 9 days. In vitro cultured CD1a⁺CD14⁻ LCs (in vitro LCs) and CD1a⁻CD14⁺ interstitial DCs (CD14⁺ DCs) were sorted, loaded with the HLA-A201-restricted melanoma peptide MART-1 (26-35) and cultured with autologous naïve CD8⁺ T cells for up to 10 days. When primed by in vitro LCs, tetramer-binding MART-1-specific CD8⁺ T cells expressed higher amounts of surface CD8 than T cells primed with CD14⁺ DCs (FIGS. 1B and 1C). In contrast, when analyzing memory responses such as influenza-specific matrix protein CD8+ T cells, the two in vitro DC subsets loaded with 1 μM of the HLA-A201-restricted Flu Matrix peptide M1 Flu-MP (58-66), were equally efficient at expanding memory CD8⁺ T cells (Klechevsky et al., 2008) and the specific CD8+ T cells expressed comparable levels of surface CD8 (FIG. 1F). To analyze cytokine secretion patterns, naïve CD8+ T cells were CFSE-labeled and cultured with the skin DC subsets for 7 days. Flow cytometry analysis revealed that Dermal CD14⁺ DCs, but not skin LCs, induced the generation of CFSE^{lo} CD8+ T cells that expressed IL-13 (13% vs. 0.7%). Both LCs and dermal CD14⁺ DCs induced CD8⁺ T cells to produce IFN-γ (FIG. 1D). To monitor the secretion of cytokines using multiplex assays, CFSE^{lo} cells were sorted and stimulated with anti-CD3 and anti-CD28 mAb for 48 hours. In concordance with the flow cytometry data, CD8⁺ T cells primed by dermal CD14⁺ DCs secreted IL-13, as well as other type 2-associated cytokines (IL-4, IL-5) and IL-10. Both LCs and dermal CD14⁺ DCs induced CD8⁺ T cells that secreted IFN-γ (FIG. 1E). Thus, dermal CD14+ DCs, but not LCs induce a fraction of naïve CD8+ T cells to differentiate into type 2 CD8+ T cells (TC2).

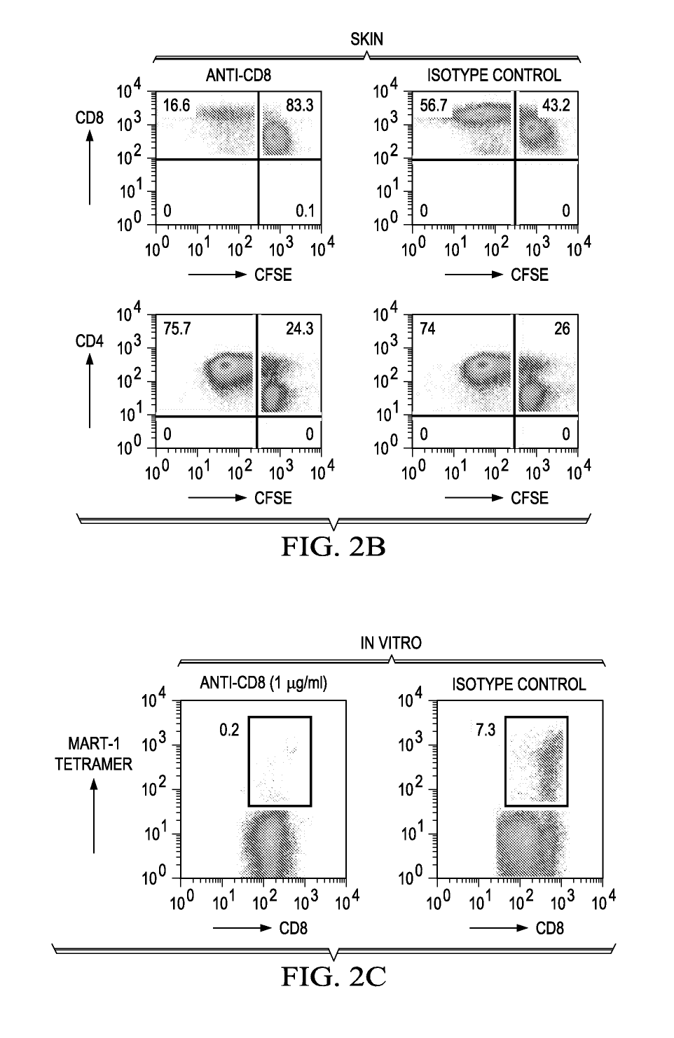

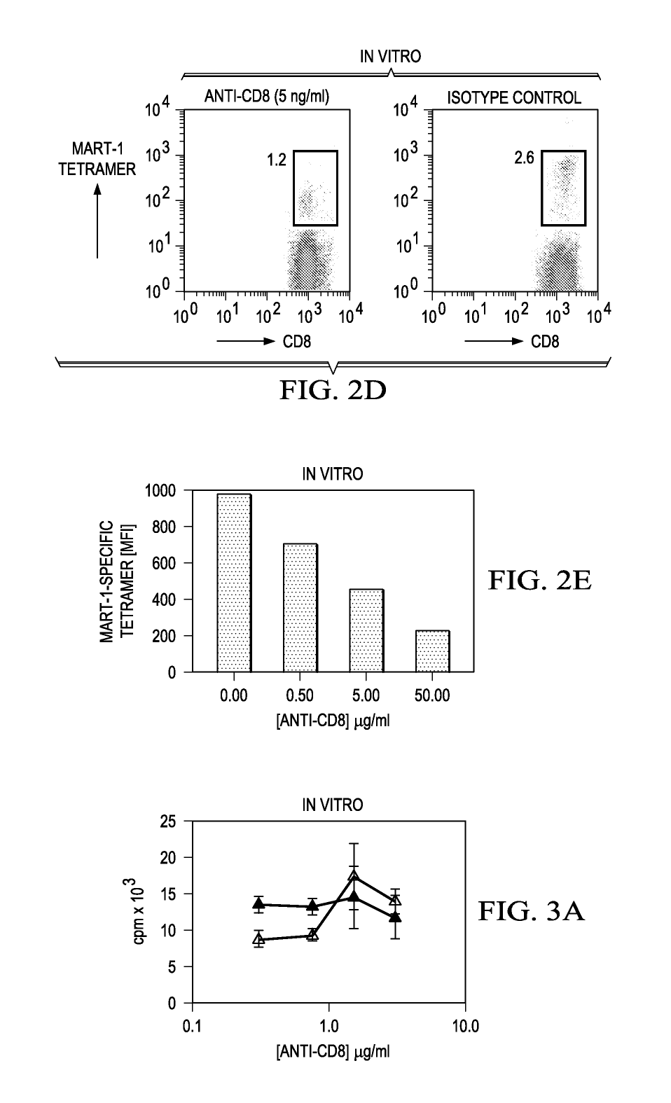

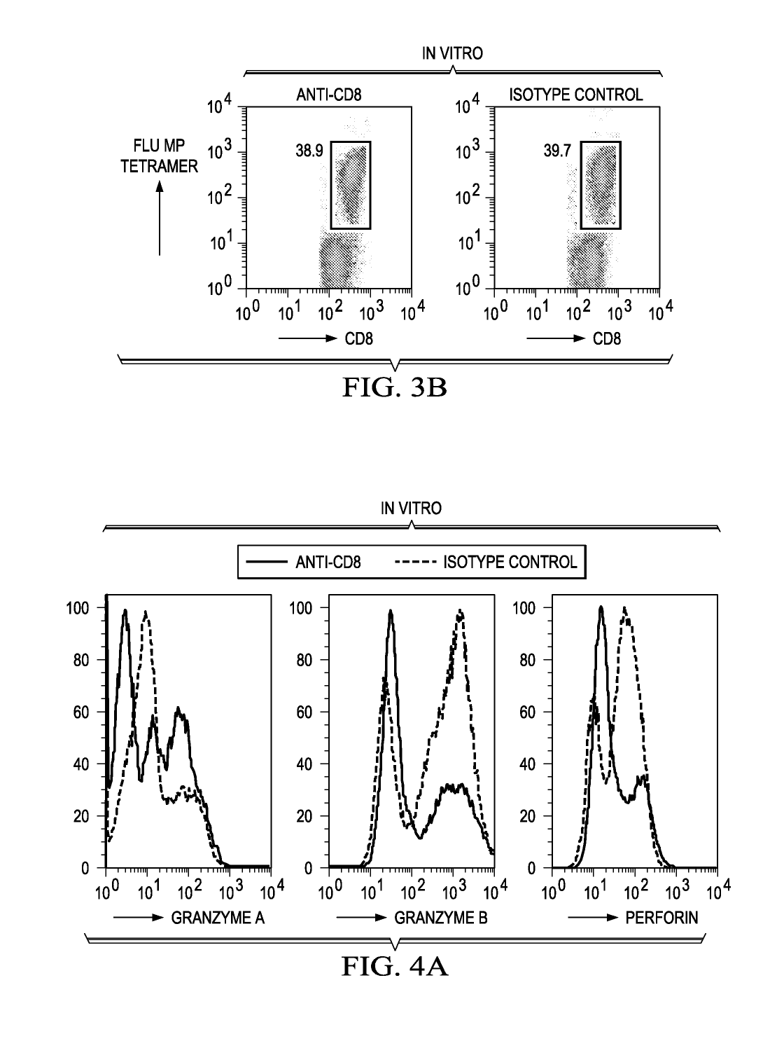

Anti-CD8 mAb inhibits priming of antigen-specific CD8⁺ T cells: The addition of anti-CD8 antibody to a mixed lymphocyte reaction (MLR) of skin LCs and naïve allogeneic T cells (FIG. 2A) resulted in ˜80% inhibition of [³H]thymidine incorporation (78%±12 reduction, n=3 p<0.0001). In MLR performed with in vitro-LCs and allogeneic naïve CD4⁺ and CD8⁺ T cells, the anti-CD8 mAbs inhibited the proliferation of CD8⁺ T cells as assessed using CFSE dilution (16.6% CFSE^{lo} vs. 56.7% CFSE^{lo}, FIG. 2B; upper panel) without affecting that of CD4⁺ T cells (75.7% CFSE^{lo} vs. 74% CFSE^{lo}, FIG. 2B; lower panel). Microscopic examination revealed that addition of anti-CD8 mAb yielded cultures composed of a few, scattered, small clusters of CD8⁺ T cells and DCs, while those performed with an isotype control showed numerous large cell clusters (not shown). The anti-CD8 mAb was also able to block the expansion of autologous MART-1-specific CD8⁺ T cells by MART-1-pulsed in vitro-LCs (FIG. 2C). Low concentrations of anti-CD8 (i.e. 5 ng/ml) to co-cultures of in vitro generated MART-1-pulsed LCs with naïve autologous CD8⁺ T cells did not inhibit the expansion of antigen-specific CD8⁺ T cells. Yet, the expanded cells bound MART-1 tetramer with a lower intensity (FIGS. 2D and 2E) thereby indicating the generation of T cells showing lower avidity. The addition of anti-CD8 mAb, at concentrations as high as 2.5 μg/ml did not inhibit the proliferation of memory alloreactive CD8⁺ T cells induced by the in vitro LCs (FIG. 3A), or of autologous Flu-MP-specific CD8⁺ T cells (FIG. 3B). Taken together these data demonstrate a critical role for CD8 in DC-mediated priming of antigen-specific CD8+ T cells.

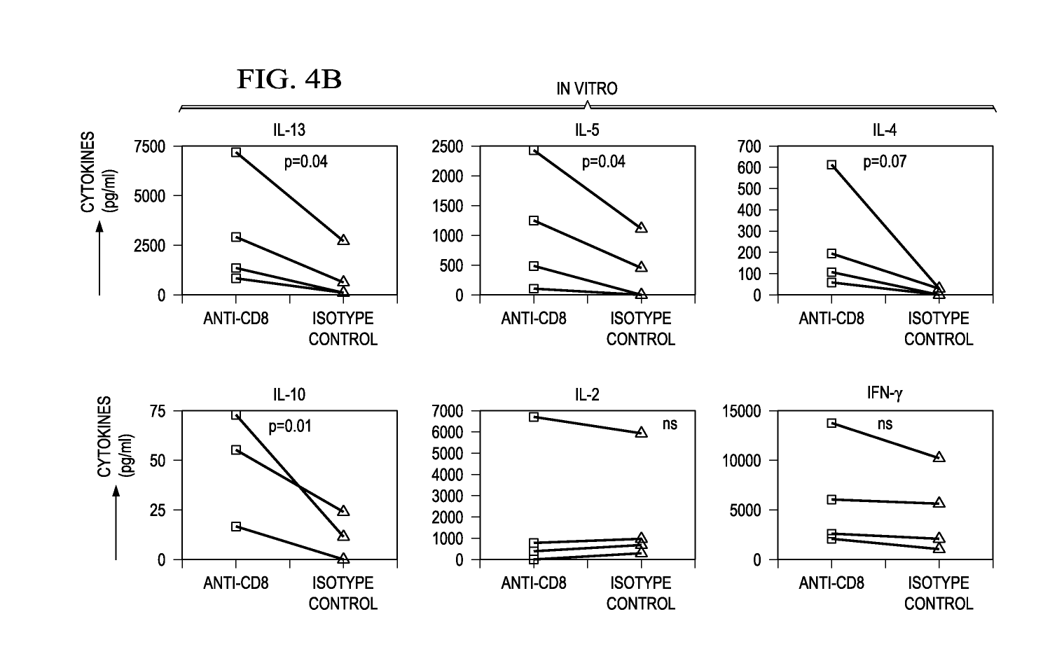

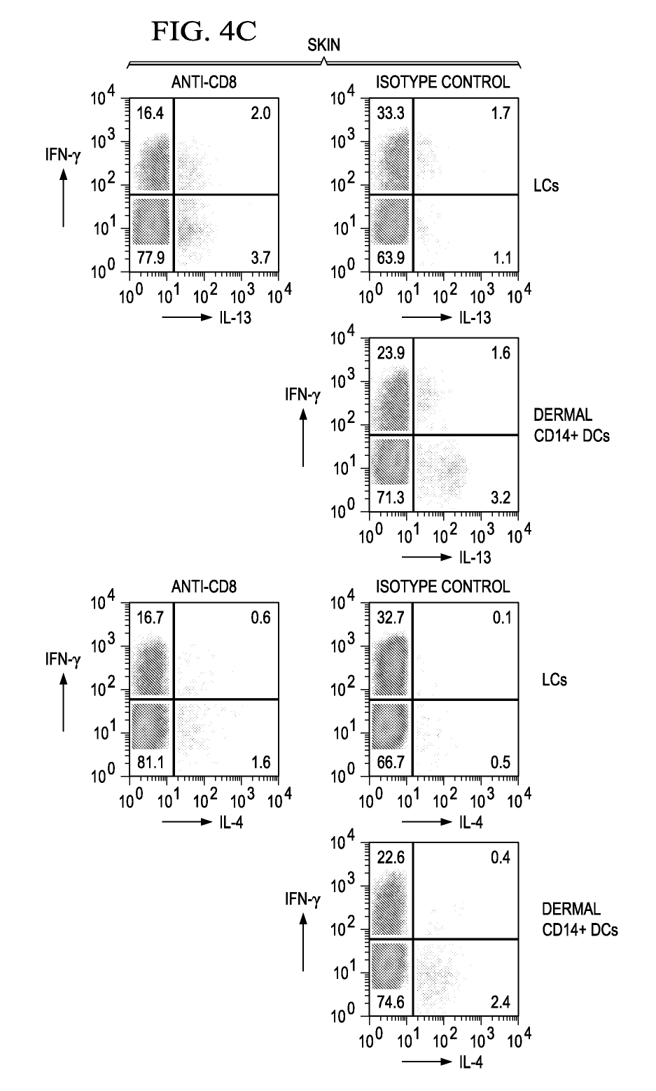

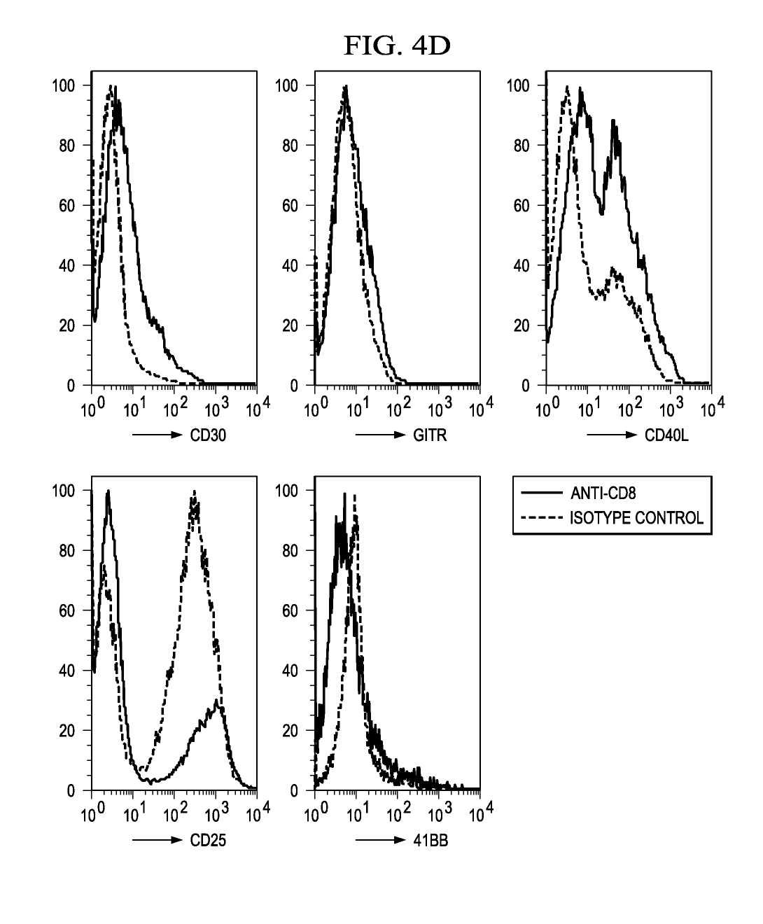

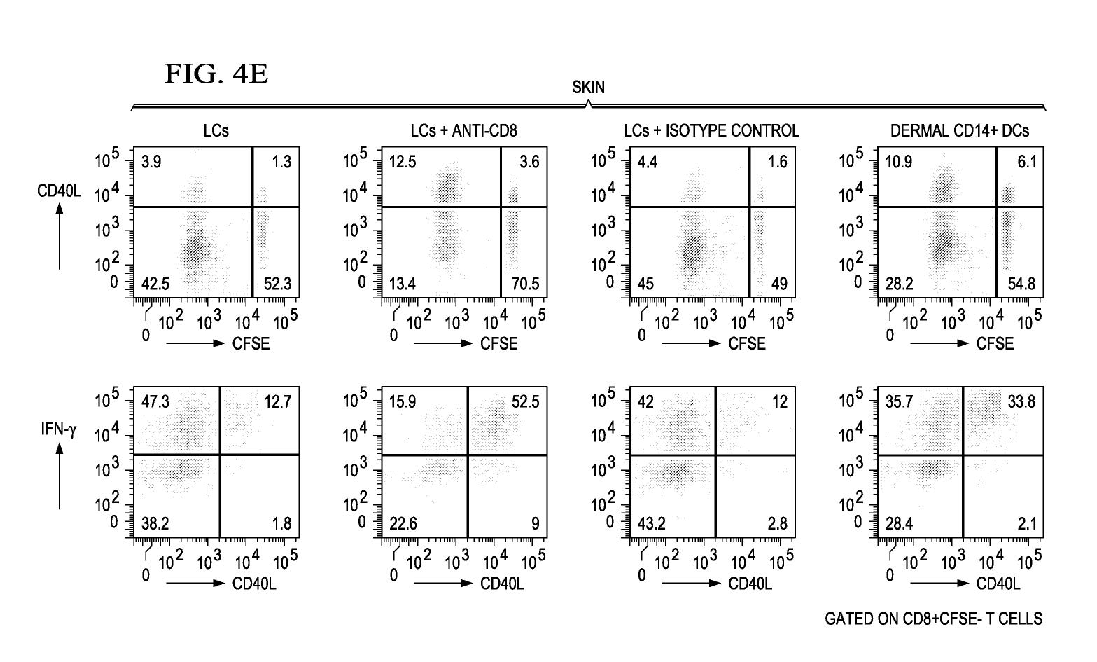

CD8⁺ T cells primed with anti-CD8 yield Type 2 T cells (TC2): As the anti-CD8 mAb only partly blocked CD8⁺ T cell proliferation, we wondered whether the remaining proliferating cells might show skewed differentiation. Indeed, addition of anti-CD8 mAb during priming of CD8+ T cells with allogeneic DCs yielded cells that expressed lower levels of the effector molecules granzyme B and perforin (FIG. 4A). To analyze cytokine secretion patterns using multiplexed cytokine assays, CFSE-labeled naïve CD8⁺ T cells were cultured with or without anti-CD8 mAb for 7 days. CFSE^{lo} cells were sorted and stimulated with anti-CD3/CD28 mAbs for 48 hours. Indeed, cells primed with anti-CD8 mAb produced higher amounts of IL-4, IL-5, IL-13 and IL-10, but similar levels of IL-2 and IFN-γ than those from control cultures (FIG. 4B). Cultures of CD8⁺ T cells primed by skin LCs with anti-CD8 mAb, contained a higher frequency of IL-13—(FIG. 3C; left panel; 5.7 (2+3.7) vs. 2.8 (1.7+1.1)) and IL-4-producing cells (FIG. 4C; right panel; 2.2 (0.6+1.6) vs. 0.6 (0.1+0.5)) than did control cultures. Culturing LCs with naïve CD8+ T cells in the presence of anti-CD8 yielded as many IL-13-, and IL-4-producing CD8⁺ T cells as did cultures of dermal CD 14⁺ DCs with naïve CD8+ T cells (FIG. 4C; 4.8 (1.6+3.2) and 2.8 (0.4+2.4), respectively). As described earlier for TC2 T cell clones (Manetti et al., 1994; Vukmanovic-Stejic et al., 2000) the CD8⁺ T cells cultured with anti-CD8 mAb expressed higher levels of surface CD30, CD40L and GITR, but lower levels of CD25 and 4-1BB (FIG. 4D) than those cultured with isotype controls. Furthermore, as observed by the dilution of CFSE dye and the expression level of CD40L in FIG. 4E, CD8⁺ T cells cultured with dermal CD14+ DCs resembled CD8+ T cells primed by LCs and anti-CD8 mAb (10.9 and 12.5 vs. 3.9 and 4.4%). Collectively, the data indicate that blocking CD8 allows the priming of some naïve CD8+ T cells into TC2.

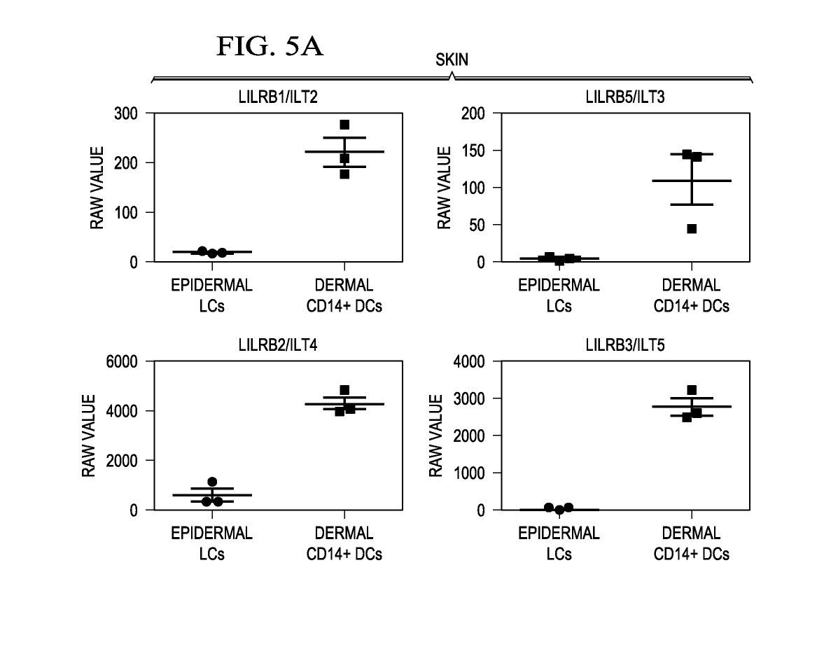

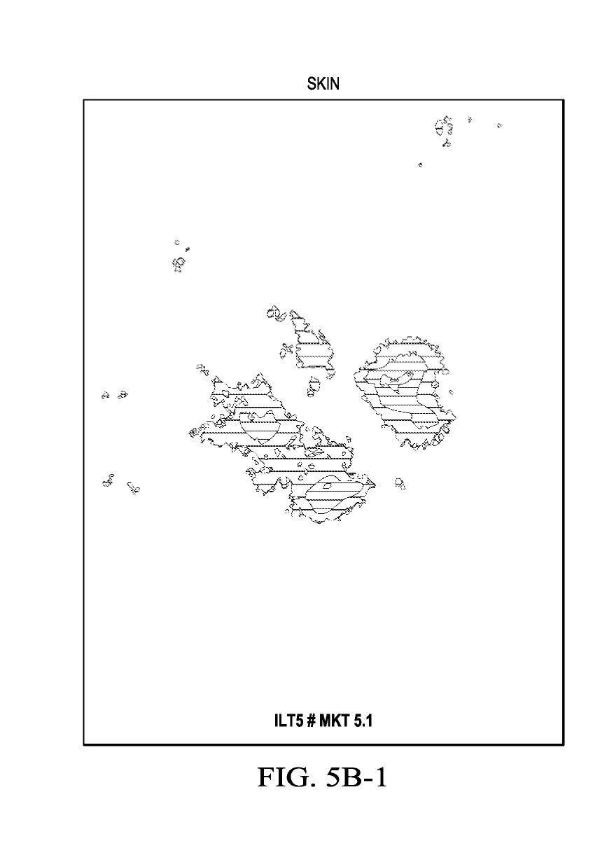

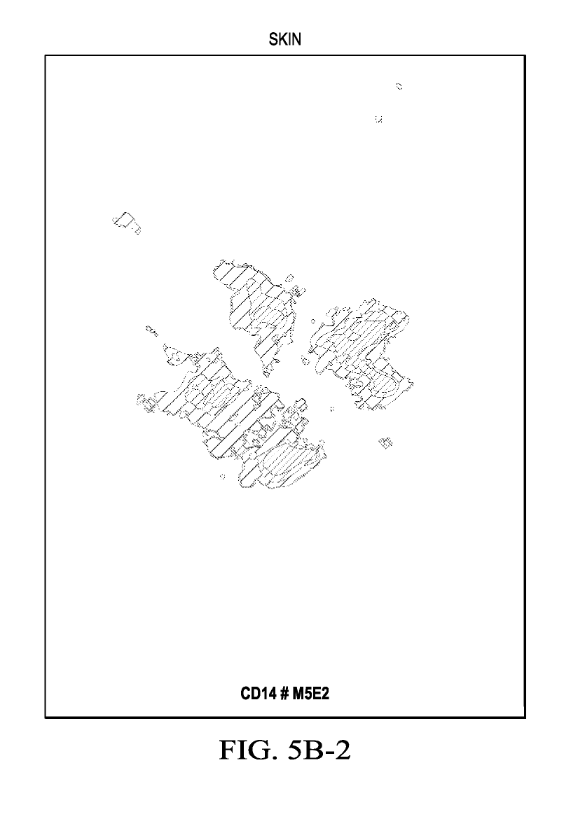

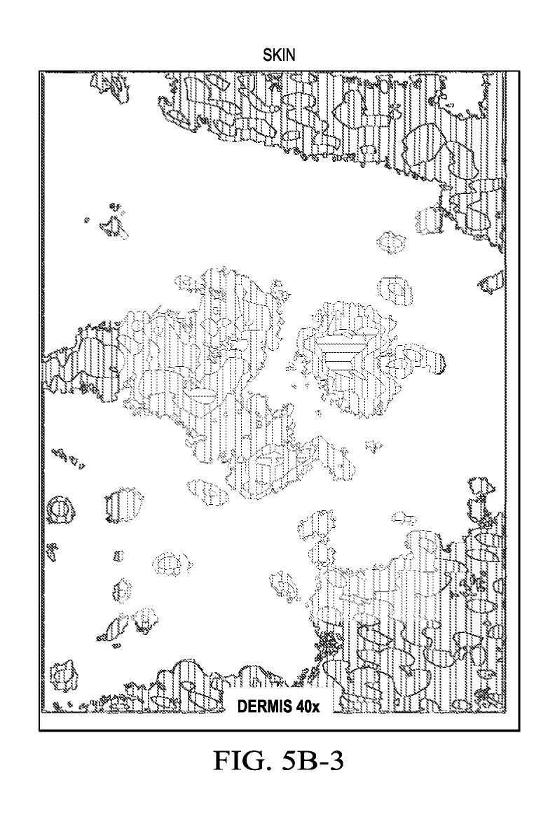

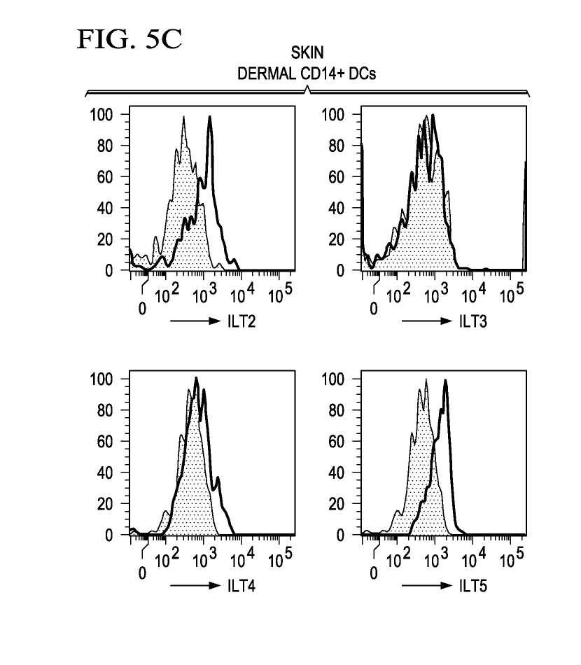

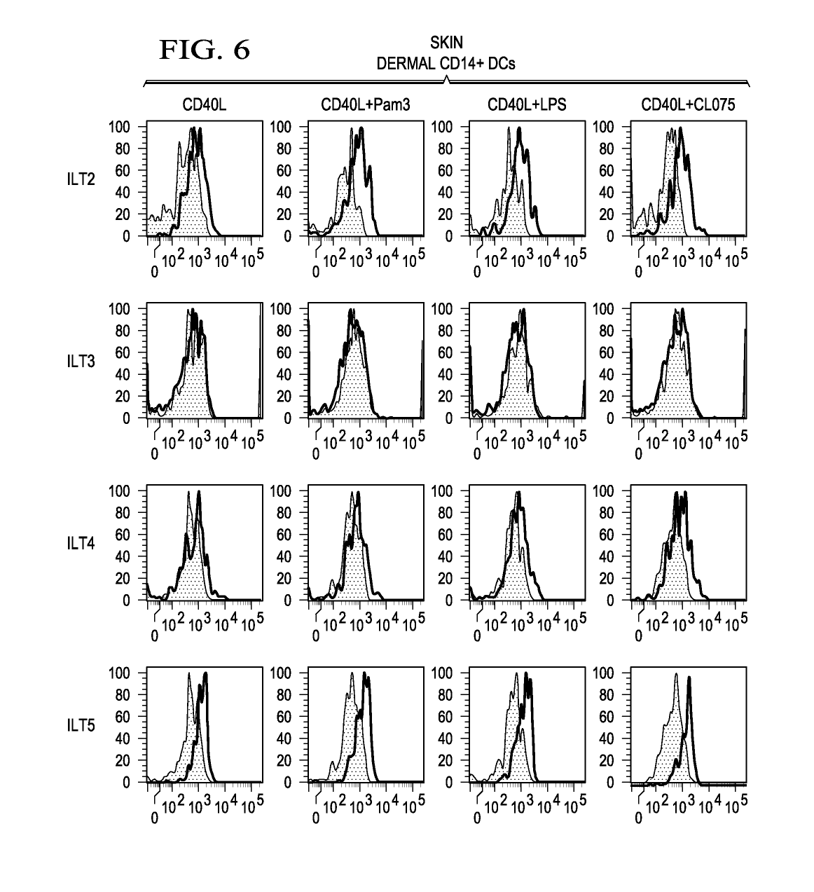

Dermal CD 14⁺ DCs, but not LCs, express ILT receptors: The above results led to hypothesis that dermal CD14⁺ DCs, but not LCs, might express CD8 antagonists such as the ILT receptors (Shiroishi et al., 2003). ILT2 and ILT4 represent such candidates as they bind both classical and non-classical MHC class I, and compete with CD8 for its binding to MHC. Microarray analysis of freshly purified skin DCs indeed revealed that dermal CD 14⁺ DCs, but not LCs, express ILT2, ILT3, ILT4 and ILT5 transcripts (FIG. 5A). Immunofluorescence staining of frozen skin sections revealed the expression of ILT5 on dermal CD14⁺ DC (FIG. 5B-1 (ILT5), 5B-2 (CD14), and 5B-3 (Dermis)). Flow cytometry analysis of DCs that migrated out of skin revealed that dermal CD14⁺ DCs express ILT2, ILT4 and ILT5 (FIG. 5C). Activation of purified DC subsets through CD40, with or without TLR agonists, did not alter their ILT receptor expression (FIG. 6). Thus, the expression ILT2, ILT4 and ILT5 is restricted to dermal CD14⁺ DC.

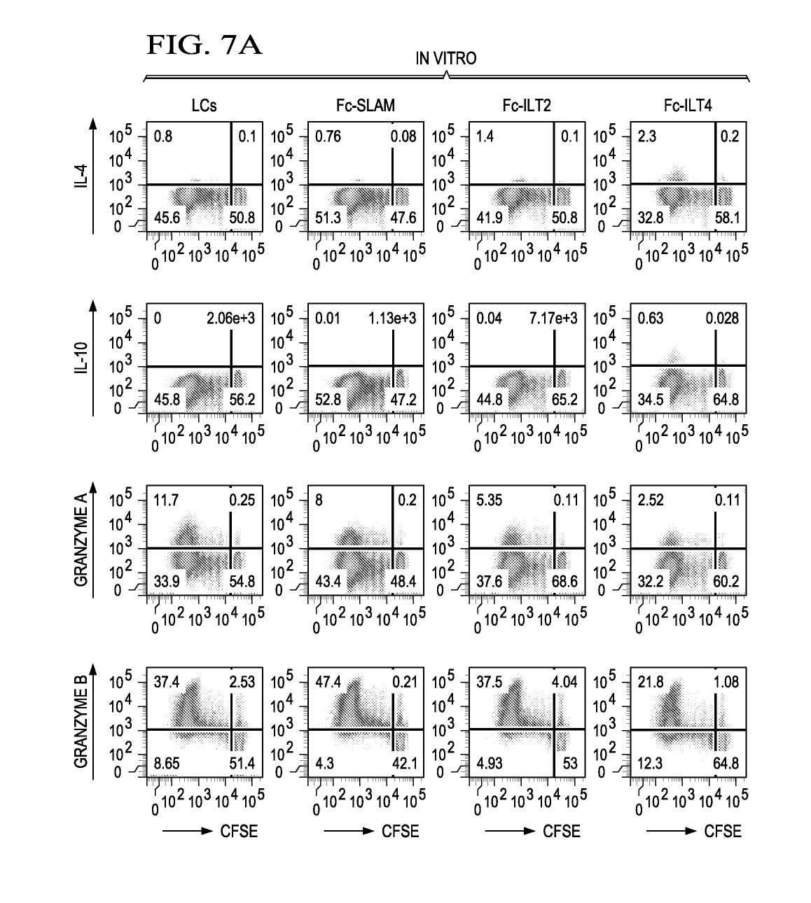

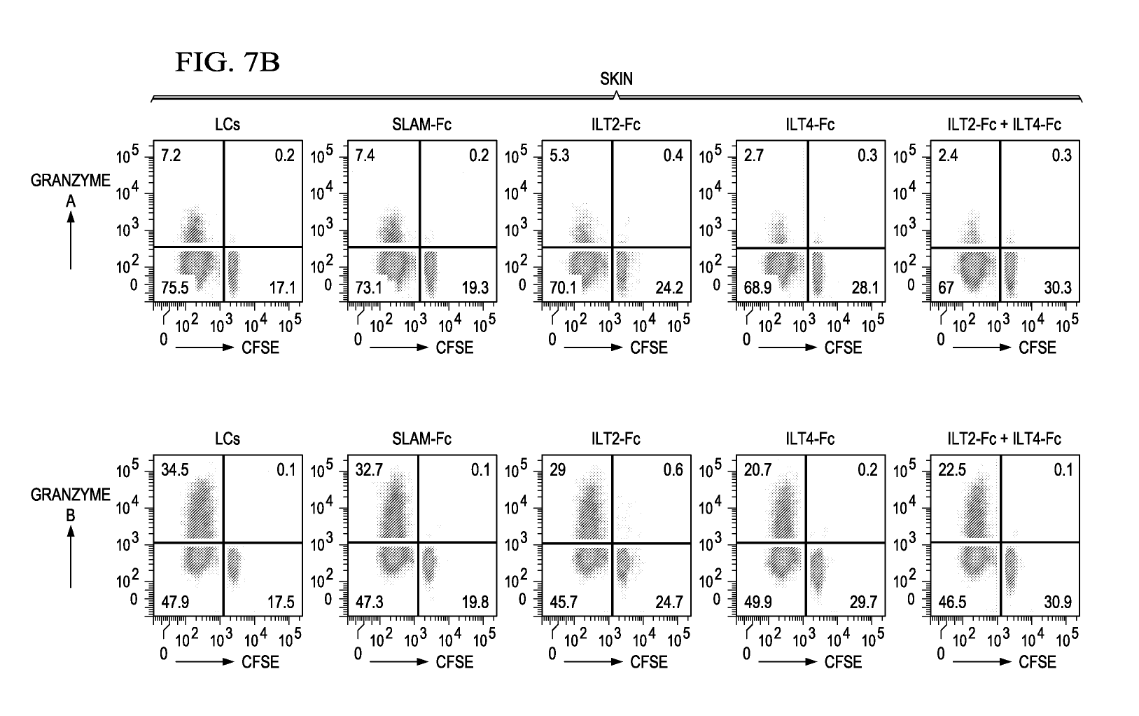

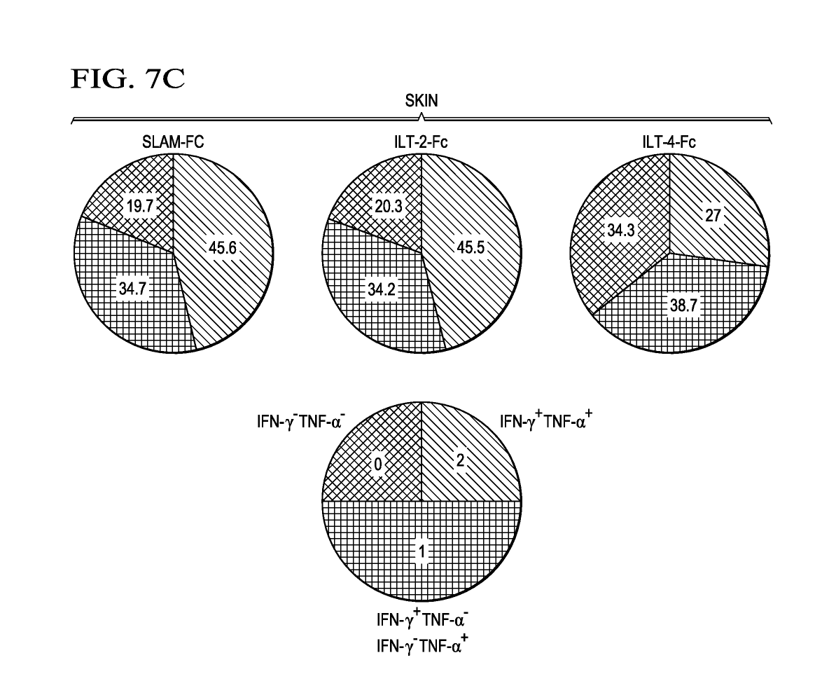

Soluble ILT2 and ILT4 inhibit the generation of multifunctional CD8⁺ T cells by LCs: To establish that ILT2 and ILT4 might actually prevent CD14⁺ DCs from generating multifunctional T cells, soluble forms of ILT2 and ILT4 proteins (extracellular domains fused to an Fc fragment), were added to co-cultures of in vitro-LCs (that do not express ILTs) and naïve CD8⁺ T cells. As shown in the two lower rows of FIG. 7A, naïve CD8⁺ T cells exposed to autologous in vitro LCs differentiate into cells expressing granzyme A and B. Addition of soluble ILT4- and to a lower extent ILT2 construct yielded T cells expressing low levels of granzymes. The lack of effect of an irrelevant Fc fusion protein (SLAM-Fc) indicates that the activity is borne by the ILT2 and ILT4 moiety rather than the Fc portion. The two upper rows of FIG. 5A shows that the addition of the ILT4- and to a lower extent ILT2 construct to the co-cultures resulted in an increased frequency of IL-4 and IL-10-producing CD8⁺ T cells. Similar results were observed when testing the soluble ILT2 and ILT4 constructs in co-cultures of skin LCs and naïve CD8 T cells (FIG. 7B). The lack of inhibition of the frequency of CFSE^{lo} cells indicates that the soluble ILT-Fc fusion proteins do not inhibit the proliferation of the CD8⁺ T cells (FIGS. 7A and 7B). Concomitantly, the ILT4 constructs induced a decreased in CD8⁺ T cells secreting both TNF-α and IFN-γ (27 vs. 45%) (FIG. 7C). Thus, soluble ILT2-Fc and ILT4-Fc inhibit the generation of effector CD8⁺ T cells induced by LCs.

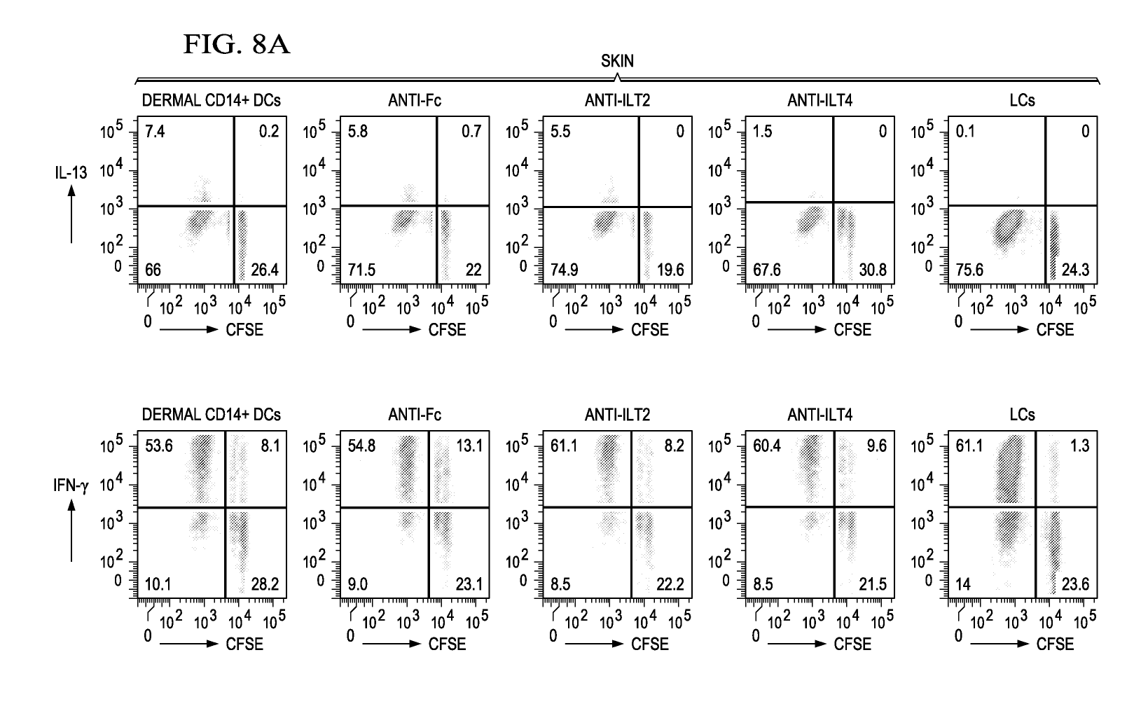

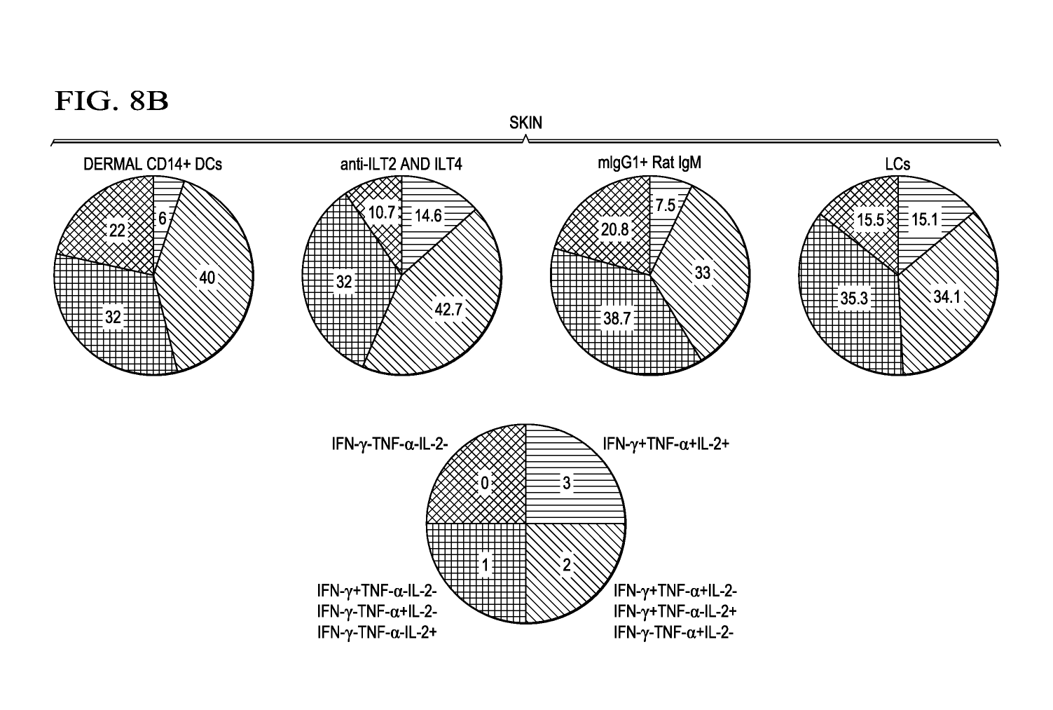

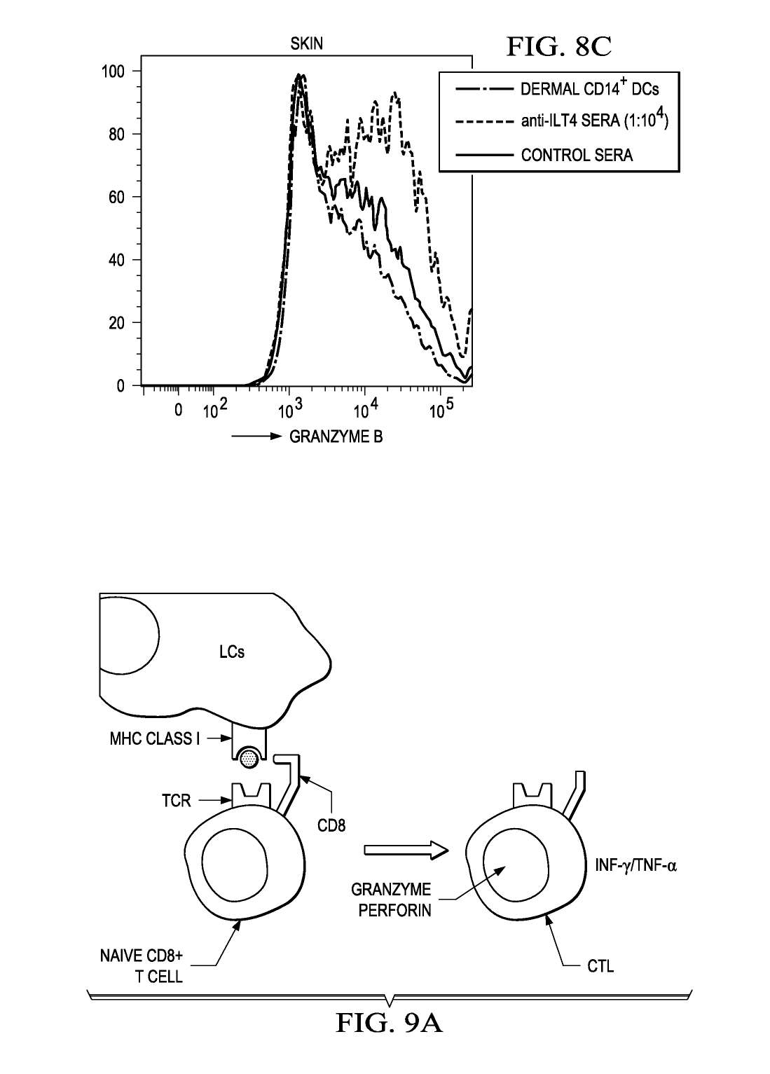

Anti-ILT2 and ILT4 mAbs enable dermal CD14+ DCs to generate multifunctional CD8⁺ T cells: The inventors used polyclonal Abs generated against ILT molecules to assess whether they would enhance the generation of effector CD8⁺ T cells by dermal CD14+ DCs by preventing the binding of ILTs to the CD8 co-receptor. To this end, mice were immunized with each soluble ILT2 and ILT4 proteins. They yielded sera that bound to the relevant ILT with some specificity (not shown). Thus, dermal CD14⁺ DCs were cultured with naïve CD8⁺ T cells and anti-ILT2 or anti-ILT4 sera diluted 1:100. Serum from mice immunized with only the Fc portion of the ILT served as controls. After 9 days, the cultured cells were stimulated with PMA and Ionomycin for 5 hours. In line with our previous study, both skin LCs (right column) and dermal CD14⁺ DCs (left column) induced naïve CD8⁺ T cells to proliferate and secrete IFN-γ, while only dermal CD14⁺ DCs-primed CD8⁺ T cells produced IL-13 (FIG. 8A). Addition of anti-ILT2, or control serum to co-cultures of dermal CD14+ DCs and naïve CD8⁺ T cells altered neither the level proliferation, nor the production of cytokines Blocking ILT4 during priming of naïve CD8+ T cells with dermal CD14⁺ DCs resulted in a reduction (from 7.4 to 1.5%) of IL-13-producing CD8⁺ T cells. Addition of a combination of anti-ILT2 and anti-ILT4 to co-cultures of dermal CD14⁺ DCs and naïve CD8⁺ T cells resulted in an increased frequency of the multifunctional CD8⁺ T cells, producing at least two of the three cytokines IFN-γ, TNF-α and IL-2 (from 6% to 14.6%) (FIG. 8B) and were similar to the levels that are observed with LCs alone (15.1%). Blocking ILT4 on dermal CD14⁺ DCs using anti-ILT4 mouse anti serum enhanced the expression of granzyme B by CFSE^{lo} cells (FIG. 8C), when compared to co-cultures performed with control serum or no serum at all (FIG. 8C). Thus, antagonists to ILT2 and ILT4 increase the capacity of dermal CD14⁺ DCs to generate polyfunctional effector CD8⁺ T cells.

DCs are composed of several subsets endowed with distinct immunological functions. The molecular basis for these functional differences remains poorly understood. The current study was initiated to understand the mechanisms that confer LCs with a stronger potency than CD14⁺ DCs for priming CD8⁺ T cell responses. Indeed, while LCs effectively expand CD8^{high} multifunctional T cells, naïve CD8⁺ T cells proliferating in response to dermal CD14⁺ DCs, display low levels of surface CD8 and production of Type 2-associated cytokines (IL-4, IL-5 and IL-13). Using allogeneic responses our study shows that limiting CD8 density on naïve T cells with anti-CD8 mAb during co-culture with LCs inhibited T cell proliferation and altered the quality of the response. This resulted in polarization towards Type 2 phenotypes associated with low frequency of granzyme- and perforin-expressing cells, low surface CD8, CD25, but high CD30 and CD40L expression (Cronin et al., 1995; Maggi et al., 1994; Manetti et al., 1994; Vukmanovic-Stejic et al., 2000). These observations supported that dermal CD14⁺ DCs might express a cell-intrinsic receptor that interferes with CD8-engagement.

Microarray analysis revealed that dermal CD14⁺ DCs expressed ILT4 and ILT2, which bind MHC Class I and can compete with CD8 on its binding site (Colonna et al., 1998; Endo et al., 2008; Shiroishi et al., 2003). Indeed, ILT receptors were shown to impair NK cell function, as well as to promote the generation of regulatory CD8⁺ and CD4+ T cell populations (Chang et al., 2002). Using soluble-ILT fusion proteins and polyclonal ILT antibodies, the inventors demonstrated the role of ILT4 and, to a lower extent, ILT2 in the regulation of multifunctional effector CD8⁺ T cells by dermal CD14⁺ DCs. Though ILT3 inhibits CD8⁺ T cell responses and promotes the induction of suppressor CD8⁺ T cells, CD8^{low} CD28⁻ population (Vlad et al., 2010; Vlad et al., 2008), our data do not support such function for dermal DCs. While dermal CD14+ DCs expressed low levels of ILT3 transcripts, the protein could not detected at steady state or upon microbial stimulation. Addition of soluble ILT3-Fc fusion protein to co-cultures of LCs and naïve CD8⁺ T cells, induced only a minor reduction in the effector CD8⁺ T cell priming. In contrast, ILT5 could be detected at high levels on the surface of purified CD14⁺ DCs, and was the only member that is detectable in situ in skin sections.

Its role in dermal CD14+ DCs remains to be established. Other inhibitory receptors such as the non-classical MHC, HLA-G that was shown to act in conjunction with ILT4, could promote the generation of IL-10-producing suppressor CD8^{low} T cells (Naji et al., 2007). Only primary, but not recall, responses against viral or allogeneic antigens were sensitive to the inhibitory effect of anti-CD8. Differences in the interaction of Lck with CD8 within naïve and memory CD8⁺ T cells might explain this finding; Indeed, while in naïve cells only a few CD8 molecules are associated with Lck, in memory and effector cells Lck is constitutively associated with the co-receptor CD8. There its interaction with CD3 components, does not require the extracellular binding of CD8 to the cognate MHC molecule (Bachmann et al., 1999; Tewari et al., 2006). Alternatively, it has been proposed that the CD8 molecule of naïve and memory cells interacts with pMHC in two distinct orientations, making the later insensitive to anti-CD8 blockage (Chang et al., 2006).

Several studies indicate a unique role for CD8 in immunoregulation, particularly in fine-tuning the activation threshold of T cells and to compensate for lower numbers of antigen-specific pMHC complexes (Feinerman et al., 2008).

Furthermore, co-engineering of CD4⁺ T cells with a melanoma-specific TCR and CD8α co receptor, resulted in a reduced production of IL-4, IL-5 and IL-10 and promoted tumor regression (Willemsen et al., 2006). The present study further supports the importance of CD8 accessibility during primary responses of naïve CD8+ T cells. In addition, the co-localization of CD8 with the T cell receptor appears to control the T cell functional immune outcome (effector function as opposed to a state of T cell anergy) (Demotte et al., 2008).

The biological role of TC2 cells remains mostly unknown though they might display a regulatory function (Salgame et al., 1991). Patient studies have revealed an expansion of TC2 populations in various disease conditions including cancer (Minkis et al., 2008; Roberts et al., 2009; Sheu et al., 2001) and viral infections such as HIV or CMV (Maggi et al., 1994; Maggi et al., 1997). In all of these cases, TC2 accumulation was associated with disease pathogenesis. In addition, CD40L- as well as IL-4-producing CD8⁺ T cells were found to have a B cell-helper function (Cronin et al., 1995; Hermann et al., 1995; Maggi et al., 1994; Nazaruk et al., 1998), which also might be concordant with CD14⁺ DCs functional specialization of controlling humoral responses (Caux et al., 1997; Klechevsky et al., 2008). Blocking CD8 can be used in clinical application, such as controlling the pathogenic effect of allogeneic CD8⁺ T cells in allograft rejection. Using this approach the patient can mount recall responses, as opposed to the general immunosuppressive treatments that increase susceptibility to viral infections.

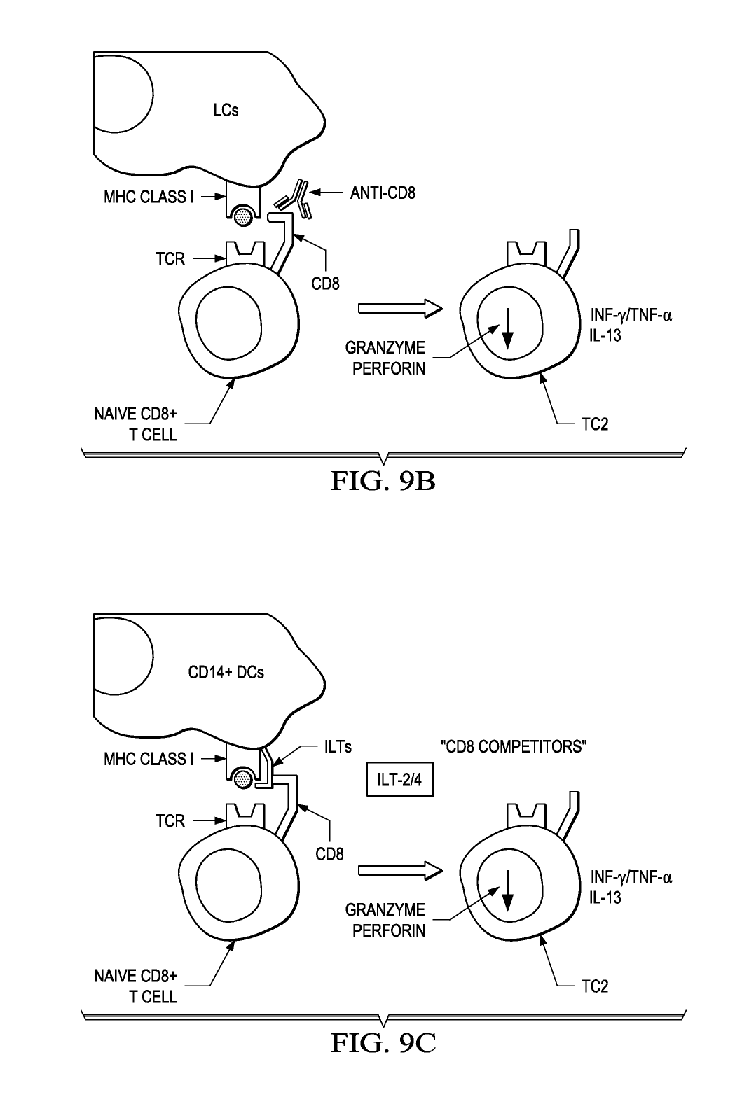

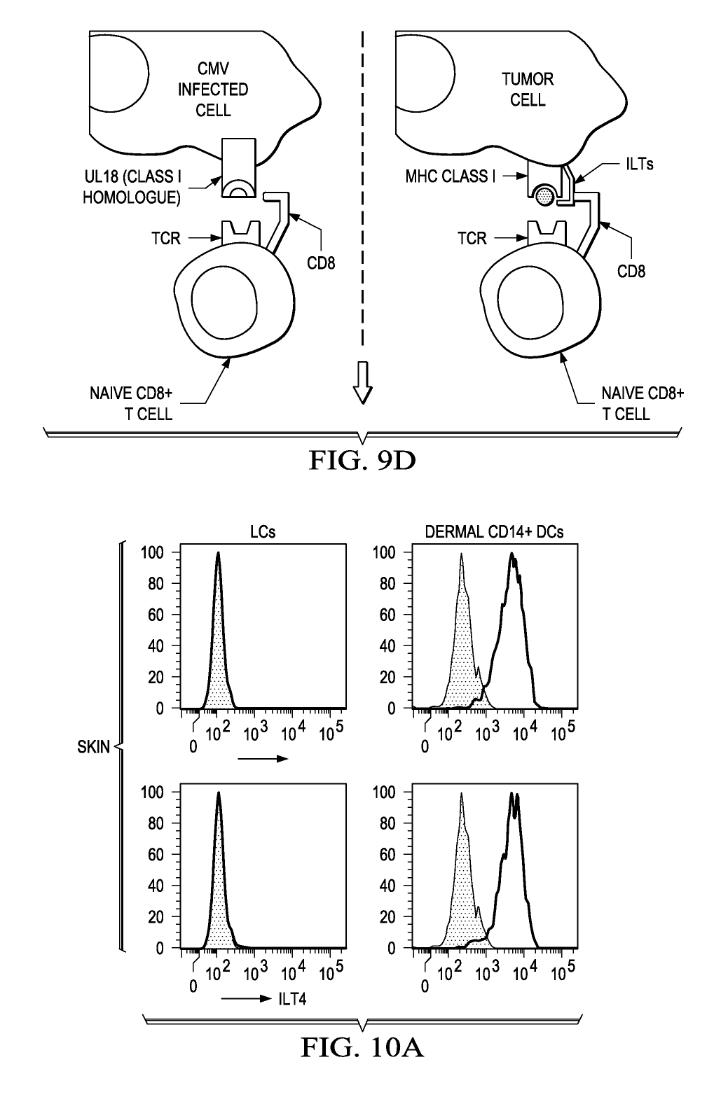

The present invention demonstrates that CD8 modulation during primary response regulates the balance between Type 1 effector and Type 2 responses. These data indicate that dermal CD14⁺ DCs harness the CD8-antagonist receptors, ILT2 and ILT4, to modulate efficient CTL differentiation and induce the generation of TC2 cells (FIG. 7). Viruses such as CMV that express class-I homologous proteins (Beck and Barrell, 1988; Yang and Bjorkman, 2008) might employ this escape mechanism to prevent induction of viral specific CD8⁺ T cell responses and thus promote TC2 cell induction with limited ability to clear the infected cells or malignant cells. Similarly, tumors might also upregulate surface ILT2 or ILT4 that can limit the induction of CTL and enhance tumor burden (FIGS. 9A-9D). Strategies to block ILT expression on DCs may be useful to augment dendritic cell function to enhance immune responses to chronic viral infections and cancer. Alternatively, mobilizing dermal CD14⁺ DCs may be a useful approach of attenuate effector responses to combat transplant rejection (Cobbold et al., 1990), and chronic inflammatory diseases.

FIGS. 9A-9D is a schematic of a model showing the action of ILT2 and ILT4 as CD8-antagonists to prevent efficient CTL priming by human dermal CD14⁺ DCs: (FIG. 9A) LCs prime high avidity polyfunctional effector CD8⁺ T cells, (FIG. 9B) Blocking CD8 during priming of naïve CD8+ T cells and LCs lead to the generation of type-2 cytokine secretion T cells. Similarly in FIG. 9C ILT2 and ILT4 receptors that are expressed by dermal CD14⁺ DCs resulting in suboptimal priming of effector CD8⁺ T cells and Type 2 cytokines, by possibly competing with CD8 on its binding to MHC class I, and (FIG. 9D) Viruses and tumor cells may utilize the ILT receptors to evade immunity.

Immunofluorescent staining of ILT on human skin. Human abdominal skins were obtained from healthy donors who underwent cosmetic surgeries at Baylor University Medical Center in accordance with Institutional Review Board guidelines. Cryo-sections were fixed in cold acetone, dried and blocked for non-specific fluorescence with Fc Receptor Block and Background Buster (Innovex, CA) and goat serum. Sections were incubated in mouse anti-CD14 (clone M5E2; 10 μg/ml) and rat anti-ILT4 (clone; 10 μg/ml) or isotype antibodies. After washing, sections were stained with goat anti-mouse AF568 (Invitrogen) and goat anti-rat AF488 (Invitrogen), and then subsequently stained with DAPI (Molecular Probes). Single channel images were acquired using the same exposures for antibody and isotype staining and identical scaling was applied. Single channels were then assigned color and overlaid. The digital images were taken using an Olympus BX51 with a Planapo20x/0.7 or Planapo40x/0.95 objective, a Roper Coolsnap HQ camera and Metamorph software vs 6.2r6 (Molecular Devices, CA).

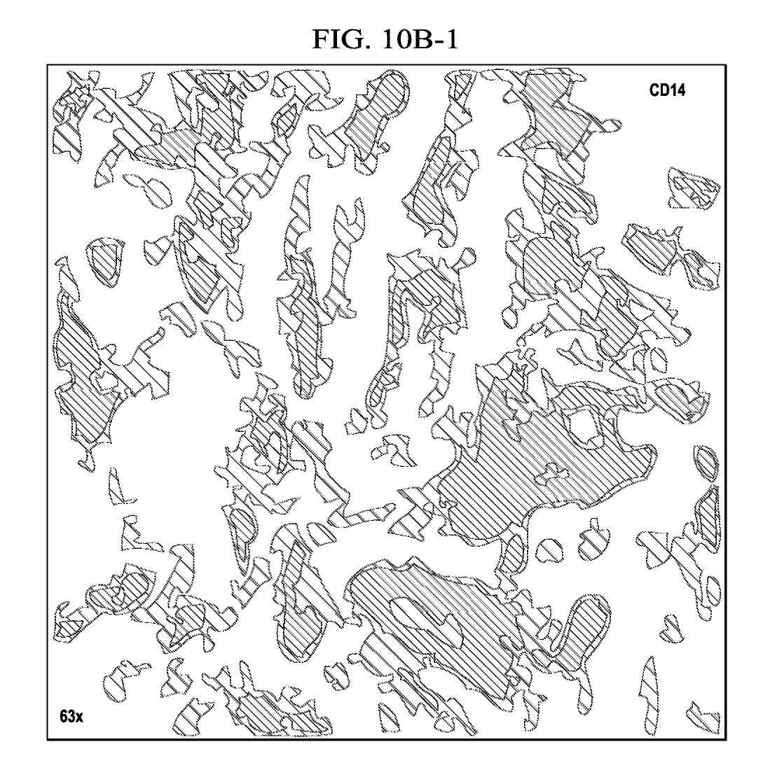

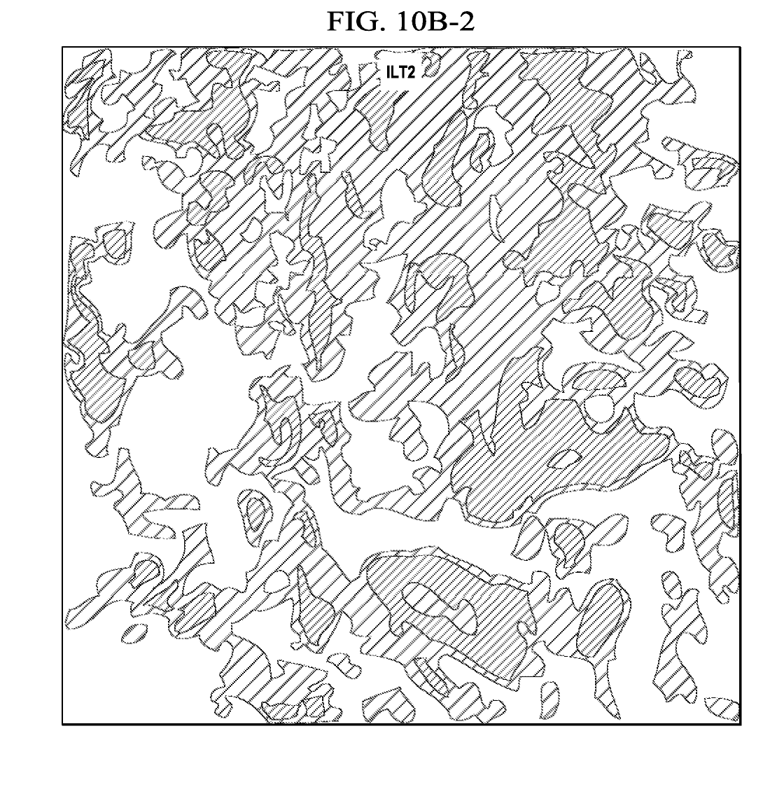

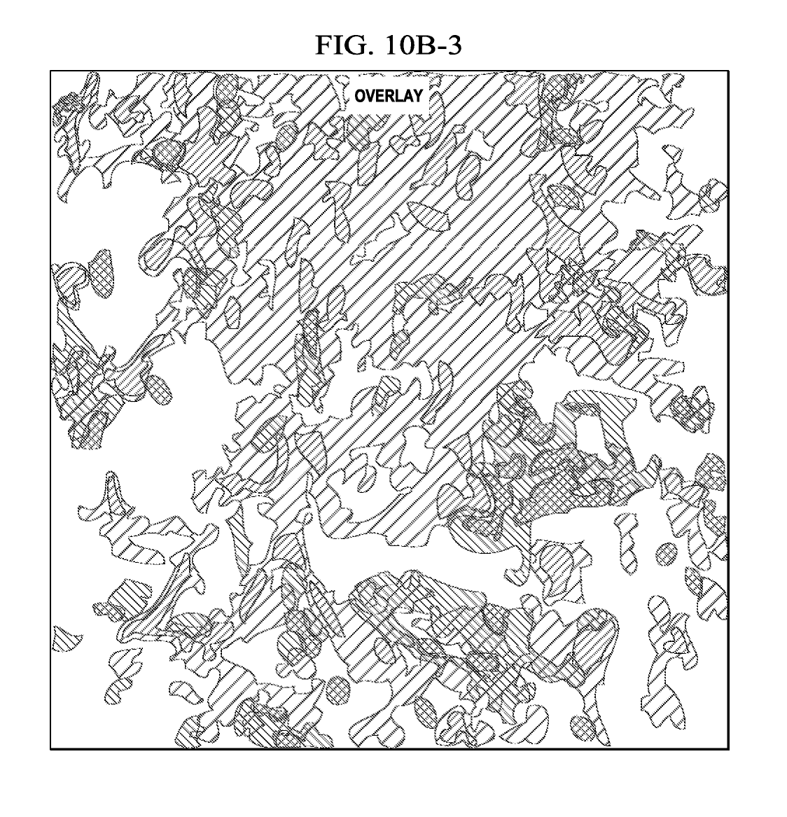

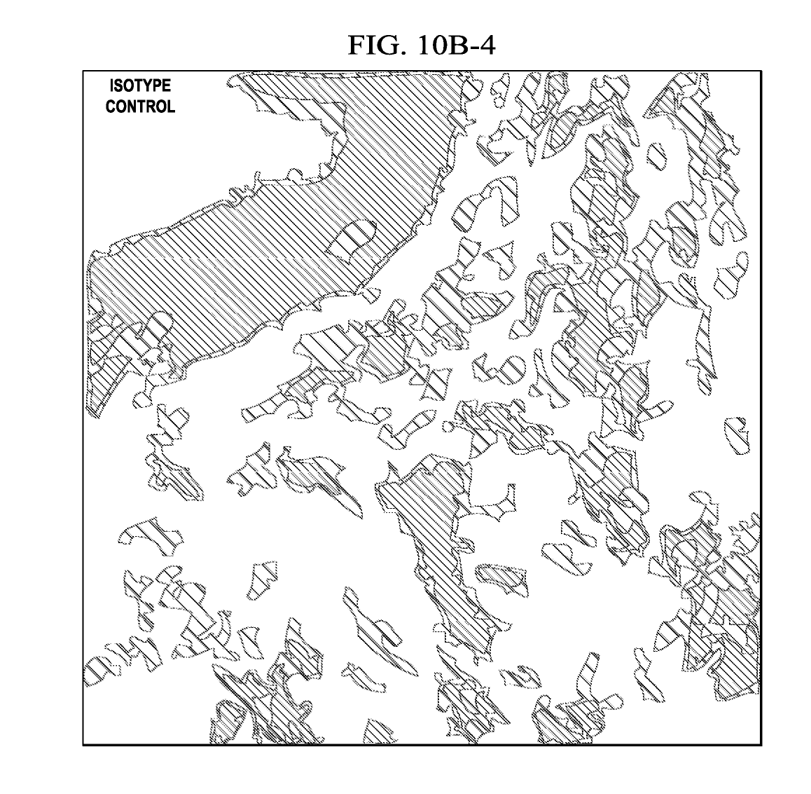

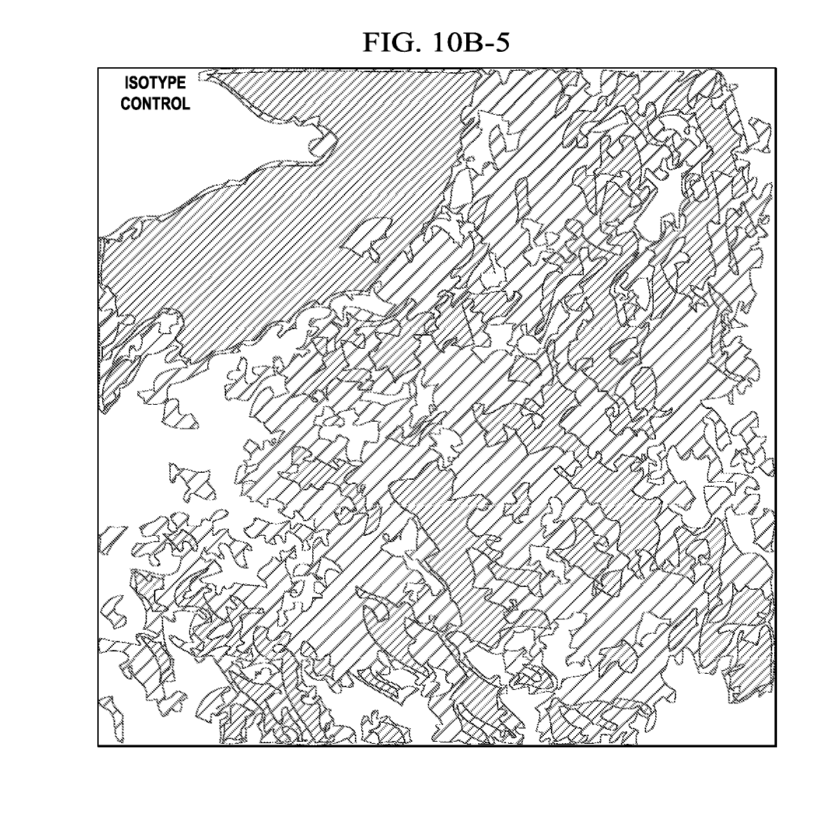

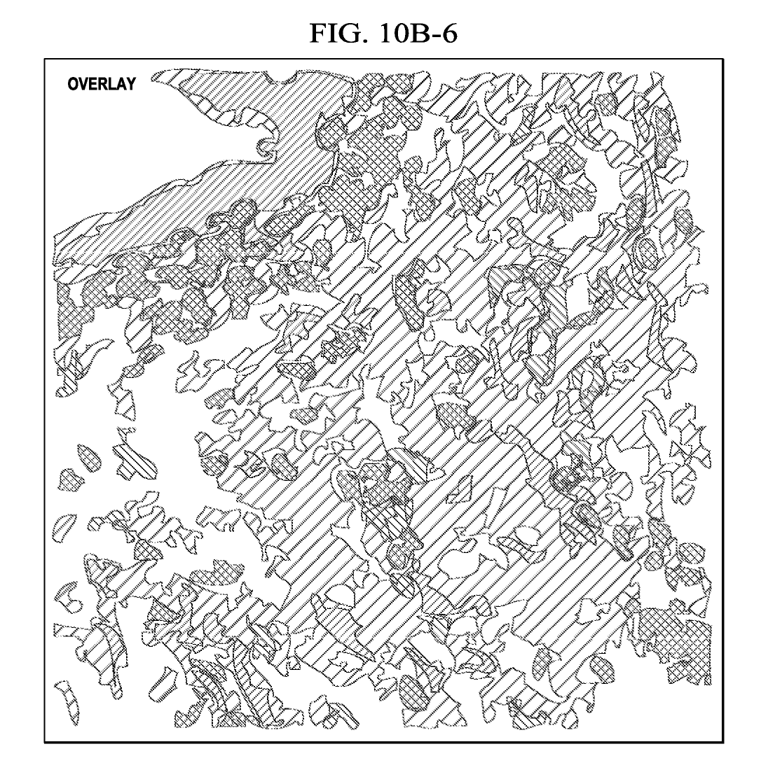

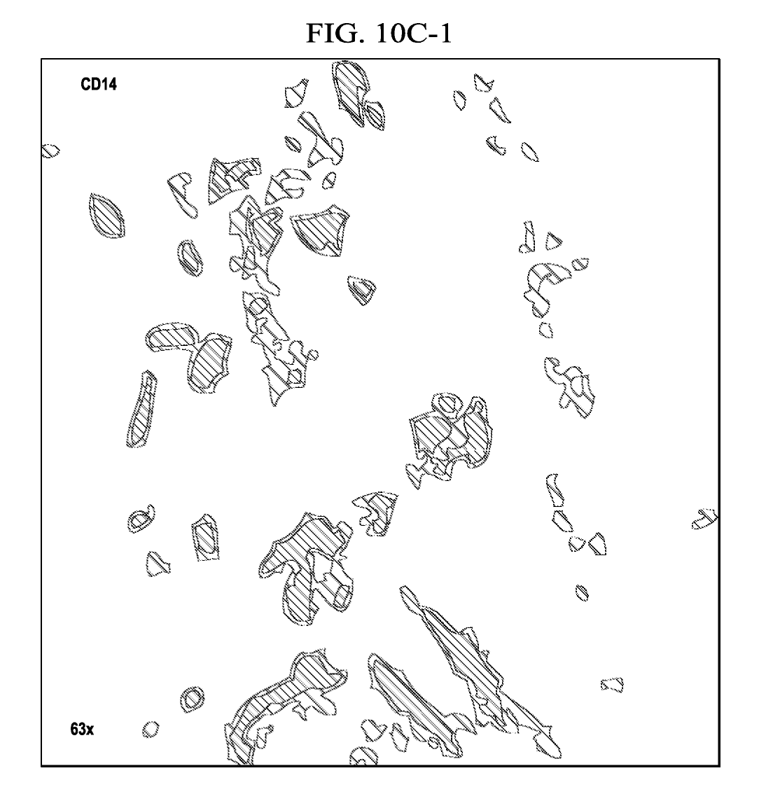

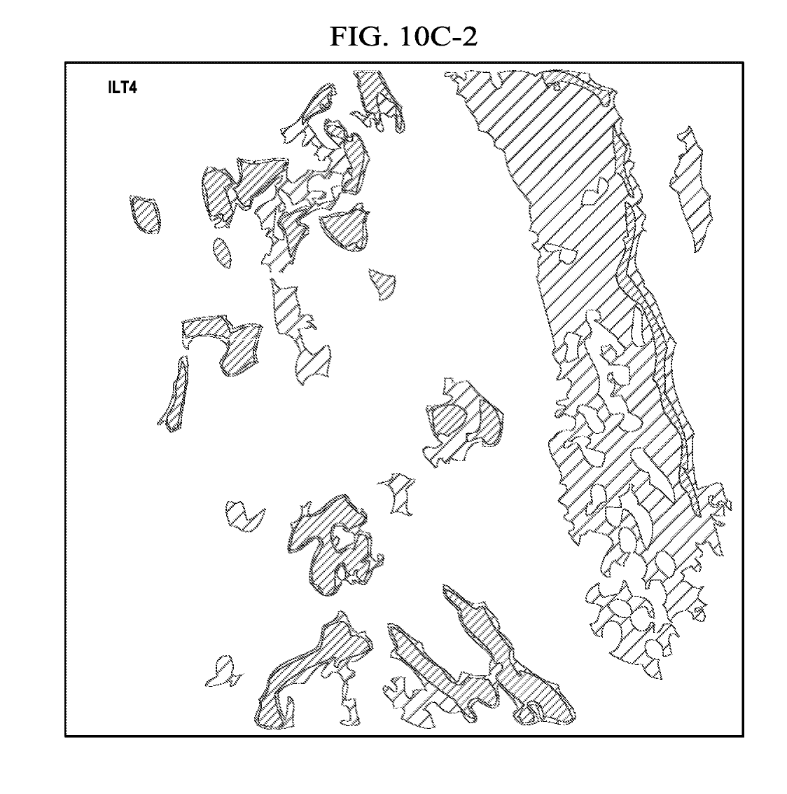

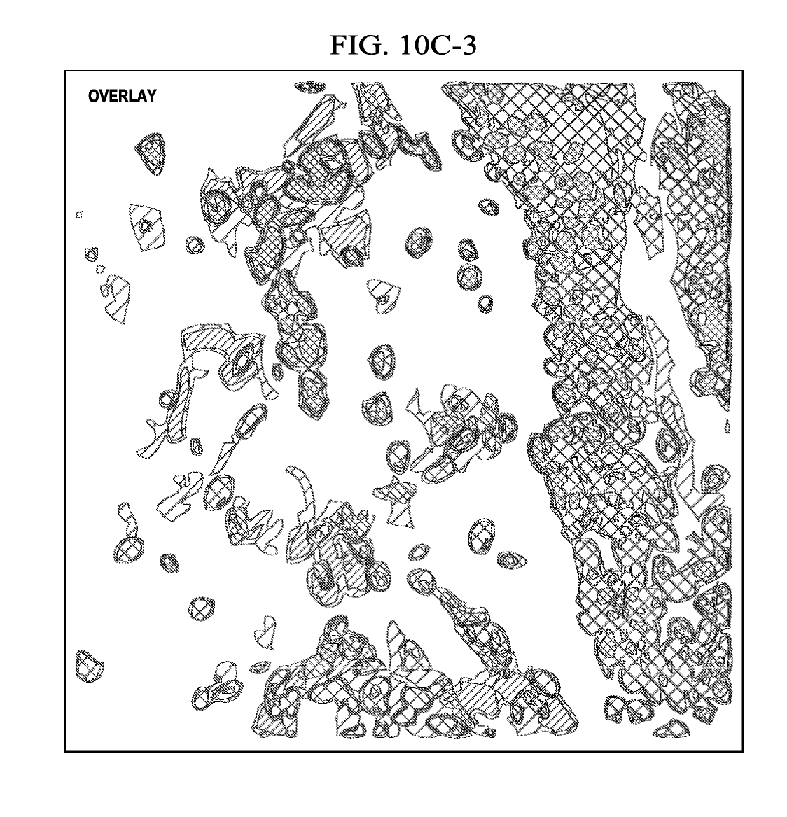

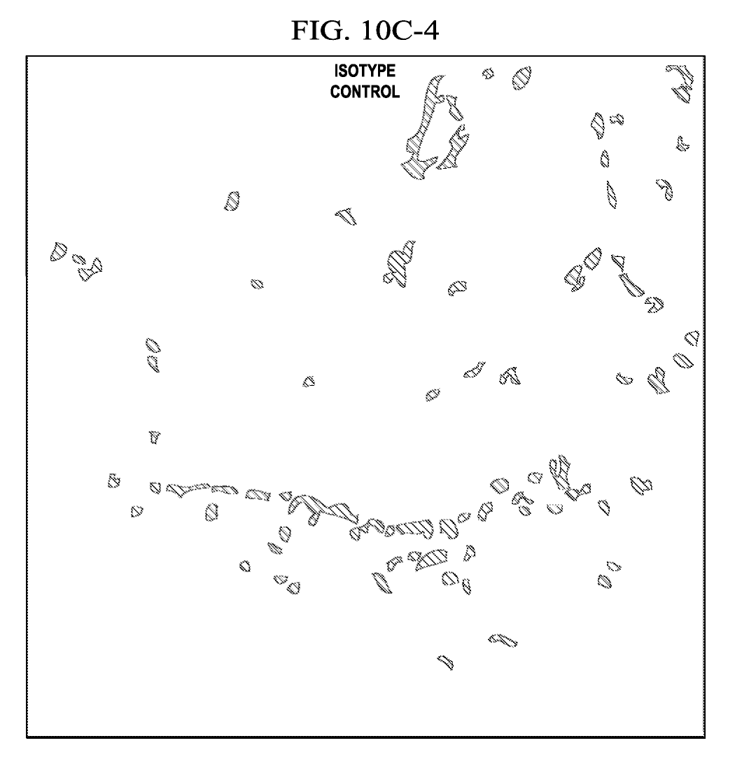

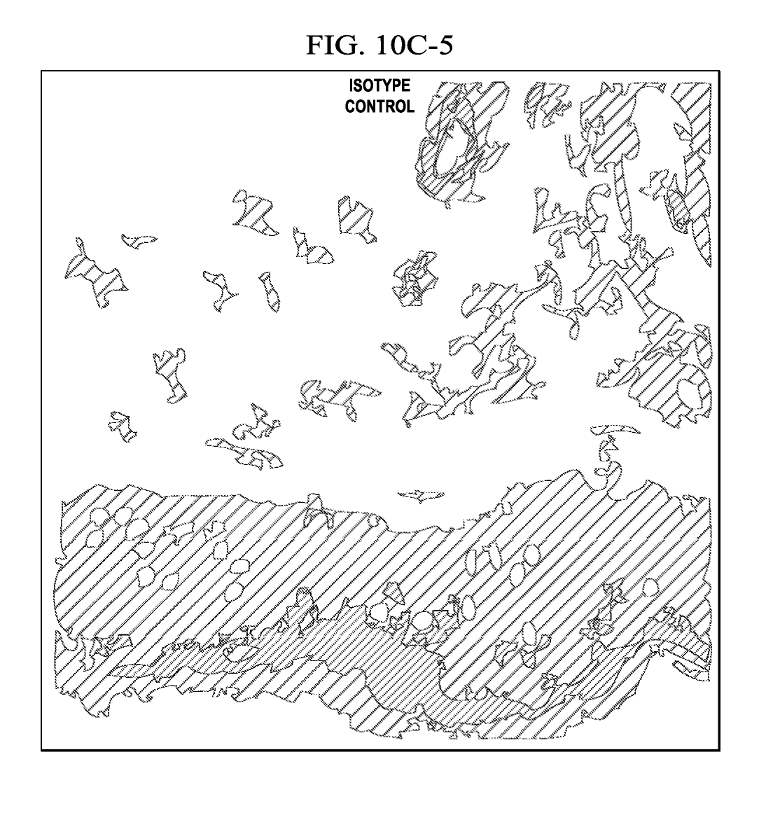

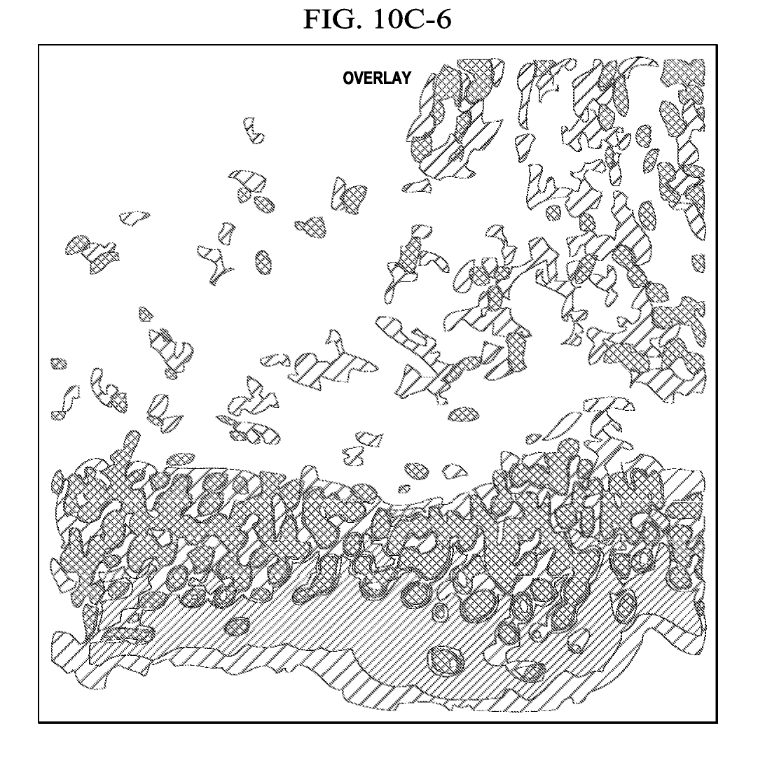

FIGS. 10A to 10C show expression analysis of the ILT family receptors by the skin DC subsets, FIG. 10A shows a flow cytometry analysis of the ILT2 and ILT4 receptors on the surface of LCs and dermal CD14⁺ DCs (black histogram), grey histogram represents isotype control. Data are representative of 4 independent studies, FIGS. 10B-1 (CD14), 10B-2 (ILT2), 10B-3 (overlay), 10B-4 (isotype control), 10B-5 (isotype control), 10B-6 (overlay) show immunofluorescence staining of ILT2, and FIGS. 10C-1 (CD14), 10C-2 (ILT4), 10C-3 (overlay), 10C-4 (isotype control), 10C-5 (isotype control), 10C-6 (overlay) show immunofluorescence staining of ILT4 receptors on sections of human dermis. ILT is visualized in green, CD14 in red and cell nuclei in blue.

It is contemplated that any embodiment discussed in this specification can be implemented with respect to any method, kit, reagent, or composition of the invention, and vice versa. Furthermore, compositions of the invention can be used to achieve methods of the invention.

It may be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it may be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Patent Publication No. 20100135997: Polypeptide Monomers and Dimers Containing Mutated ILT.
U.S. Patent Publication No. 20110034675: ILT3 Binding Molecules and uses Therefor.
U.S. Pat. No. 6,180,600: CD8 Antagonists.
Apte, S. H., Groves, P., Olver, S., Baz, A., Doolan, D. L., Kelso, A., and Kienzle, N. (2010). IFN-gamma inhibits IL-4-induced type 2 cytokine expression by CD8 T cells in vivo and modulates the anti-tumor response. J Immunol 185, 998-1004.
Bachmann, M. F., Gallimore, A., Linkert, S., Cerundolo, V., Lanzavecchia, A., Kopf, M., and Viola, A. (1999). Developmental regulation of Lck targeting to the CD8 coreceptor controls signaling in naive and memory T cells. J Exp Med 189, 1521-1530.
Banchereau, J., and Steinman, R. M. (1998). Dendritic cells and the control of immunity. Nature 392, 245-252.
Beck, S., and Barrell, B. G. (1988). Human cytomegalovirus encodes a glycoprotein homologous to MHC class-I antigens. Nature 331, 269-272.
Caux, C., Massacrier, C., Vanbervliet, B., Dubois, B., Durand, I., Cella, M., Lanzavecchia, A., and Banchereau, J. (1997). CD34+ hematopoietic progenitors from human cord blood differentiate along two independent dendritic cell pathways in response to granulocyte-macrophage colony-stimulating factor plus tumor necrosis factor alpha: II. Functional analysis. Blood 90, 1458-1470.
Cella, M., Dohring, C., Samaridis, J., Dessing, M., Brockhaus, M., Lanzavecchia, A., and Colonna, M. (1997). A novel inhibitory receptor (ILT3) expressed on monocytes, macrophages, and dendritic cells involved in antigen processing. J Exp Med 185, 1743-1751.
Chang, C. C., Ciubotariu, R., Manavalan, J. S., Yuan, J., Colovai, A. I., Piazza, F., Lederman, S., Colonna, M., Cortesini, R., Dalla-Favera, R., and Suciu-Foca, N. (2002). Tolerization of dendritic cells by T(S) cells: the crucial role of inhibitory receptors ILT3 and ILT4. Nat Immunol 3, 237-243.
Chang, H. C., Tan, K., and Hsu, Y. M. (2006). CD8alphabeta has two distinct binding modes of interaction with peptide-major histocompatibility complex class I. J Biol Chem 281, 28090-28096.
Cobbold, S. P., Martin, G., and Waldmann, H. (1990). The induction of skin graft tolerance in major histocompatibility complex-mismatched or primed recipients: primed T cells can be tolerized in the periphery with anti-CD4 and anti-CD8 antibodies. Eur J Immunol 20, 2747-2755.
Colonna, M., Navarro, F., Bellon, T., Llano, M., Garcia, P., Samaridis, J., Angman, L., Cella, M., and Lopez-Botet, M. (1997). A common inhibitory receptor for major histocompatibility complex class I molecules on human lymphoid and myelomonocytic cells. J Exp Med 186, 1809-1818.
Colonna, M., Samaridis, J., Cella, M., Angman, L., Allen, R. L., O'Callaghan, C. A., Dunbar, R., Ogg, G. S., Cerundolo, V., and Rolink, A. (1998). Human myelomonocytic cells express an inhibitory receptor for classical and nonclassical MHC class I molecules. J Immunol 160, 3096-3100.
Connolly, J. M., Potter, T. A., Wormstall, E. M., and Hansen, T. H. (1988). The Lyt-2 molecule recognizes residues in the class 1 alpha 3 domain in allogeneic cytotoxic T cell responses. J Exp Med 168, 325-341.
Croft, M., Carter, L., Swain, S. L., and Dutton, R. W. (1994). Generation of polarized antigen-specific CD8 effector populations: reciprocal action of interleukin (IL)-4 and IL-12 in promoting type 2 versus type 1 cytokine profiles. J Exp Med 180, 1715-1728.
Cronin, D. C., 2nd, Stack, R., and Fitch, F. W. (1995). IL-4-producing CD8+ T cell clones can provide B cell help. J Immunol 154, 3118-3127.
Demotte, N., Stroobant, V., Courtoy, P. J., Van Der Smissen, P., Colau, D., Luescher, I. F., Hivroz, C., Nicaise, J., Squifflet, J. L., Mourad, M., et al. (2008). Restoring the association of the T cell receptor with CD8 reverses anergy in human tumor-infiltrating lymphocytes. Immunity 28, 414-424.
Dietrich, J., Nakajima, H., and Colonna, M. (2000). Human inhibitory and activating Ig-like receptors which modulate the function of myeloid cells. Microbes Infect 2, 323-329.
Dobrzanski, M. J., Reome, J. B., Hylind, J. C., and Rewers-Felkins, K. A. (2006). CD8-mediated type 1 antitumor responses selectively modulate endogenous differentiated and nondifferentiated T cell localization, activation, and function in progressive breast cancer. J Immunol 177, 8191-8201.
Dubois, B., Massacrier, C., Vanbervliet, B., Fayette, J., Briere, F., Banchereau, J., and Caux, C. (1998). Critical role of IL-12 in dendritic cell-induced differentiation of naive B lymphocytes. J Immunol 161, 2223-2231.
Dudziak, D., Kamphorst, A. O., Heidkamp, G. F., Buchholz, V. R., Trumpfheller, C., Yamazaki, S., Cheong, C., Liu, K., Lee, H. W., Park, C. G., et al. (2007). Differential antigen processing by dendritic cell subsets in vivo. Science 315, 107-111.
Endo, S., Sakamoto, Y., Kobayashi, E., Nakamura, A., and Takai, T. (2008). Regulation of cytotoxic T lymphocyte triggering by PIR-B on dendritic cells. Proc Natl Acad Sci USA 105, 14515-14520.
Feinerman, O., Veiga, J., Dorfman, J. R., Germain, R. N., and Altan-Bonnet, G. (2008). Variability and robustness in T cell activation from regulated heterogeneity in protein levels. Science 321, 1081-1084.
Fung-Leung, W. P., Louie, M. C., Limmer, A., Ohashi, P. S., Ngo, K., Chen, L., Kawai, K., Lacy, E., Loh, D. Y., and Mak, T. W. (1993). The lack of CD8 alpha cytoplasmic domain resulted in a dramatic decrease in efficiency in thymic maturation but only a moderate reduction in cytotoxic function of CD8+ T lymphocytes. Eur J Immunol 23, 2834-2840.
Fung-Leung, W. P., Schilham, M. W., Rahemtulla, A., Kundig, T. M., Vollenweider, M., Potter, J., van Ewijk, W., and Mak, T. W. (1991). CD8 is needed for development of cytotoxic T cells but not helper T cells. Cell 65, 443-449.
Garcia, K. C., Scott, C. A., Brunmark, A., Carbone, F. R., Peterson, P. A., Wilson, I. A., and Teyton, L. (1996). CD8 enhances formation of stable T-cell receptor/MHC class I molecule complexes. Nature 384, 577-581.
Grakoui, A., Bromley, S. K., Sumen, C., Davis, M. M., Shaw, A. S., Allen, P. M., and Dustin, M. L. (1999). The immunological synapse: a molecular machine controlling T cell activation. Science 285, 221-227.
Gregori, S., Magnani, C. F., and Roncarolo, M. G. (2009). Role of human leukocyte antigen-G in the induction of adaptive type 1 regulatory T cells. Hum Immunol 70, 966-969.
Gregori, S., Tomasoni, D., Pacciani, V., Scirpoli, M., Battaglia, M., Magnani, C. F., Hauben, E., and Roncarolo, M. G. (2010). Differentiation of type 1 T regulatory cells (Tr1) by tolerogenic DC-10 requires the IL-10-dependent ILT4/HLA-G pathway. Blood 116, 935-944.
Hermann, P., Van-Kooten, C., Gaillard, C., Banchereau, J., and Blanchard, D. (1995). CD40 ligand-positive CD8+ T cell clones allow B cell growth and differentiation. Eur J Immunol 25, 2972-2977.
Joffre, O., Nolte, M. A., Sporri, R., and Reis e Sousa, C. (2009). Inflammatory signals in dendritic cell activation and the induction of adaptive immunity. Immunol Rev 227, 234-247.
Klechevsky, E., Flamar, A. L., Cao, Y., Blanck, J. P., Liu, M., O'Bar, A., Agouna-Deciat, O., Klucar, P., Thompson-Snipes, L., Zurawski, S., et al. (2010). Cross-priming CD8+ T cells by targeting antigens to human dendritic cells through DCIR. Blood 116, 1685-1697.
Klechevsky, E., Liu, M., Morita, R., Banchereau, R., Thompson-Snipes, L., Palucka, A. K., Ueno, H., and Banchereau, J. (2009). Understanding human myeloid dendritic cell subsets for the rational design of novel vaccines. Hum Immunol 70, 281-288.
Klechevsky, E., Morita, R., Liu, M., Cao, Y., Coquery, S., Thompson-Snipes, L., Briere, F., Chaussabel, D., Zurawski, G., Palucka, A. K., et al. (2008). Functional specializations of human epidermal langerhans cells and CD 14+ dermal dendritic cells. Immunity 29, 497-510.
Le Gros, G., Ben-Sasson, S. Z., Seder, R., Finkelman, F. D., and Paul, W. E. (1990). Generation of interleukin 4 (IL-4)-producing cells in vivo and in vitro: IL-2 and IL-4 are required for in vitro generation of IL-4-producing cells. J Exp Med 172, 921-929.
Liu, Y. J. (2005). IPC: professional type 1 interferon-producing cells and plasmacytoid dendritic cell precursors. Annu Rev Immunol 23, 275-306.
Maggi, E., Giudizi, M. G., Biagiotti, R., Annunziato, F., Manetti, R., Piccinni, M. P., Parronchi, P., Sampognaro, S., Giannarini, L., Zuccati, G., and Romagnani, S. (1994). Th2-like CD8+ T cells showing B cell helper function and reduced cytolytic activity in human immunodeficiency virus type 1 infection. J Exp Med 180, 489-495.
Maggi, E., Manetti, R., Annunziato, F., Cosmi, L., Giudizi, M. G., Biagiotti, R., Galli, G., Zuccati, G., and Romagnani, S. (1997). Functional characterization and modulation of cytokine production by CD8+ T cells from human immunodeficiency virus-infected individuals. Blood 89, 3672-3681.
Maldonado-Lopez, R., De Smedt, T., Michel, P., Godfroid, J., Pajak, B., Heirman, C., Thielemans, K., Leo, O., Urbain, J., and Moser, M. (1999). CD8alpha+ and CD8alpha− subclasses of dendritic cells direct the development of distinct T helper cells in vivo. J Exp Med 189, 587-592.
Manetti, R., Annunziato, F., Biagiotti, R., Giudizi, M. G., Piccinni, M. P., Giannarini, L., Sampognaro, S., Parronchi, P., Vinante, F., Pizzolo, G., et al. (1994). CD30 expression by CD8+ T cells producing type 2 helper cytokines Evidence for large numbers of CD8+CD30+ T cell clones in human immunodeficiency virus infection. J Exp Med 180, 2407-2411.
Merad, M., Ginhoux, F., and Collin, M. (2008). Origin, homeostasis and function of Langerhans cells and other langerin-expressing dendritic cells. Nat Rev Immunol 8, 935-947.
Minkis, K., Kavanagh, D. G., Alter, G., Bogunovic, D., O'Neill, D., Adams, S., Pavlick, A., Walker, B. D., Brockman, M. A., Gandhi, R. T., and Bhardwaj, N. (2008). Type 2 Bias of T cells expanded from the blood of melanoma patients switched to type 1 by IL-12p70 mRNA-transfected dendritic cells. Cancer Res 68, 9441-9450.
Mosmann, T. R., Cherwinski, H., Bond, M. W., Giedlin, M. A., and Coffman, R. L. (1986). Two types of murine helper T cell clone. I. Definition according to profiles of lymphokine activities and secreted proteins. J Immunol 136, 2348-2357.
Mosmann, T. R., and Coffman, R. L. (1989). TH1 and TH2 cells: different patterns of lymphokine secretion lead to different functional properties. Annu Rev Immunol 7, 145-173.
Naji, A., Le Rond, S., Durrbach, A., Krawice-Radanne, I., Creput, C., Daouya, M., Caumartin, J., LeMaoult, J., Carosella, E. D., and Rouas-Freiss, N. (2007). CD3+CD4low and CD3+CD8low are induced by HLA-G: novel human peripheral blood suppressor T-cell subsets involved in transplant acceptance. Blood 110, 3936-3948.
Nakayama, K., Nakayama, K., Negishi, I., Kuida, K., Louie, M. C., Kanagawa, O., Nakauchi, H., and Loh, D. Y. (1994). Requirement for CD8 beta chain in positive selection of CD8-lineage T cells. Science 263, 1131-1133.
Nazaruk, R. A., Rochford, R., Hobbs, M. V., and Cannon, M. J. (1998). Functional diversity of the CD8(+) T-cell response to Epstein-Barr virus (EBV): implications for the pathogenesis of EBV-associated lymphoproliferative disorders. Blood 91, 3875-3883.
Nestle, F. O., Zheng, X. G., Thompson, C. B., Turka, L. A., and Nickoloff, B. J. (1993). Characterization of dermal dendritic cells obtained from normal human skin reveals phenotypic and functionally distinctive subsets. J Immunol 151, 6535-6545.
Pulendran, B., Smith, J. L., Caspary, G., Brasel, K., Pettit, D., Maraskovsky, E., and Maliszewski, C. R. (1999). Distinct dendritic cell subsets differentially regulate the class of immune response in vivo. Proc Natl Acad Sci USA 96, 1036-1041.
Ratzinger, G., Baggers, J., de Cos, M. A., Yuan, J., Dao, T., Reagan, J. L., Munz, C., Heller, G., and Young, J. W. (2004). Mature human Langerhans cells derived from CD34+ hematopoietic progenitors stimulate greater cytolytic T lymphocyte activity in the absence of bioactive IL-12p70, by either single peptide presentation or cross-priming, than do dermal-interstitial or monocyte-derived dendritic cells. J Immunol 173, 2780-2791.
Roberts, W. K., Deluca, I. J., Thomas, A., Fak, J., Williams, T., Buckley, N., Dousmanis, A. G., Posner, J. B., and Darnell, R. B. (2009). Patients with lung cancer and paraneoplastic Hu syndrome harbor HuD-specific type 2 CD8+ T cells. J Clin Invest 119, 2042-2051.
Sakaguchi, S., Miyara, M., Costantino, C. M., and Hafler, D. A. (2010). FOXP3+ regulatory T cells in the human immune system. Nat Rev Immunol 10, 490-500.
Salgame, P., Abrams, J. S., Clayberger, C., Goldstein, H., Convit, J., Modlin, R. L., and Bloom, B. R. (1991). Differing lymphokine profiles of functional subsets of human CD4 and CD8 T cell clones. Science 254, 279-282.
Salter, R. D., Benjamin, R. J., Wesley, P. K., Buxton, S. E., Garrett, T. P., Clayberger, C., Krensky, A. M., Norment, A. M., Littman, D. R., and Parham, P. (1990). A binding site for the T-cell co-receptor CD8 on the alpha 3 domain of HLA-A2. Nature 345, 41-46.
Seder, R. A., Boulay, J. L., Finkelman, F., Barbier, S., Ben-Sasson, S. Z., Le Gros, G., and Paul, W. E. (1992). CD8+ T cells can be primed in vitro to produce IL-4. J Immunol 148, 1652-1656.
Sheu, B. C., Lin, R. H., Lien, H. C., Ho, H. N., Hsu, S. M., and Huang, S. C. (2001). Predominant Th2/Tc2 polarity of tumor-infiltrating lymphocytes in human cervical cancer. J Immunol 167, 2972-2978.
Shiroishi, M., Tsumoto, K., Amano, K., Shirakihara, Y., Colonna, M., Braud, V. M., Allan, D. S., Makadzange, A., Rowland-Jones, S., Willcox, B., et al. (2003). Human inhibitory receptors Ig-like transcript 2 (ILT2) and ILT4 compete with CD8 for MHC class I binding and bind preferentially to HLA-G. Proc Natl Acad Sci USA 100, 8856-8861.
Shortman, K., and Liu, Y. J. (2002). Mouse and human dendritic cell subtypes. Nature Rev Immunol 2, 151-161.
Steinman, L. (2007). A brief history of T(H)17, the first major revision in the T(H)1/T(H)2 hypothesis of T cell-mediated tissue damage. Nat Med 13, 139-145.
Steinman, R. M., and Banchereau, J. (2007). Taking dendritic cells into medicine. Nature 449, 419-426.
Tacken, P. J., and Figdor, C. G. (2011). Targeted antigen delivery and activation of dendritic cells in vivo: Steps towards cost effective vaccines. Semin Immunol 23, 12-20.
Tewari, K., Walent, J., Svaren, J., Zamoyska, R., and Suresh, M. (2006). Differential requirement for Lck during primary and memory CD8+ T cell responses. Proc Natl Acad Sci USA 103, 16388-16393.
Vlad, G., Chang, C. C., Colovai, A. I., Vasilescu, E. R., Cortesini, R., and Suciu-Foca, N. (2010). Membrane and soluble ILT3 are critical to the generation of T suppressor cells and induction of immunological tolerance. Int Rev Immunol 29, 119-132.
Vlad, G., Cortesini, R., and Suciu-Foca, N. (2008). CD8+ T suppressor cells and the ILT3 master switch. Hum Immunol 69, 681-686.
Vukmanovic-Stejic, M., Vyas, B., Gorak-Stolinska, P., Noble, A., and Kemeny, D. M. (2000). Human Tc1 and Tc2/Tc0 CD8 T-cell clones display distinct cell surface and functional phenotypes. Blood 95, 231-240.
Willemsen, R. A., Sebestyen, Z., Ronteltap, C., Berrevoets, C., Drexhage, J., and Debets, R. (2006). CD8 alpha coreceptor to improve TCR gene transfer to treat melanoma: down-regulation of tumor-specific production of IL-4, IL-5, and IL-10. J Immunol 177, 991-998.
Woodland, D. L., and Dutton, R. W. (2003). Heterogeneity of CD4(+) and CD8(+) T cells. Curr Opin Immunol 15, 336-342.
Yang, Z., and Bjorkman, P. J. (2008). Structure of UL18, a peptide-binding viral MHC mimic, bound to a host inhibitory receptor. Proc Natl Acad Sci USA 105, 10095-10100.
Zaba, L. C., Fuentes-Duculan, J., Steinman, R. M., Krueger, J. G., and Lowes, M. A. (2007). Normal human dermis contains distinct populations of CD11c+BDCA-1+ dendritic cells and CD163+FXIIIA+ macrophages. J Clin Invest 117, 2517-2525.
Zhu, J., Yamane, H., and Paul, W. E. (2010). Differentiation of effector CD4 T cell populations (*). Annu Rev Immunol 28, 445-489.

What is claimed is:

1. An immunostimulatory composition comprising:
one or more antigenic peptides, wherein the antigenic peptides are representative of one or more epitopes of the one or more antigens implicated or involved in a disease or a condition against which the immune response, the prophylaxis, the therapy, or any combination thereof is desired; and
at least one immunoglobulin-like transcript (ILT) receptor antagonist, wherein the ILT receptor is selected from the group consisting of ILT2, ILT4, ILT5, or any combinations thereof obtained from one or more dermal CD14⁺ dendritic cells (DCs).

2. The composition of claim 1, wherein the one or more antigenic peptides is further defined as a conjugate, wherein the conjugate comprises the antigenic peptide loaded, recombinantly linked or coupled chemically or with a recombinant linker to a dendritic cell (DC)-specific antibody selected from an antibody that specifically binds to MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, and ASGPR.

3. The composition of claim 2, wherein the DC-specific antibody is humanized.

4. The composition of claim 1, wherein the antigenic peptides comprise at least one of a peptide or protein selected from gag, pol, env, nef protein, reverse transcriptase, PSA-tetramer, a HIVgag-derived p24-PLA HIV gag p24 (gag), and other HIV components, hepatitis viral antigens, influenza viral antigens and peptides selected from the group consisting of hemagglutinin, neuraminidase, Influenza A Hemagglutinin HA-1 from a H1N1 Flu strain, HLA-A201-FluMP (58-66) peptide tetramer, and Avian Flu (HA5-1), measles viral antigens, rubella viral antigens, rotaviral antigens, cytomegaloviral antigens, respiratory syncytial viral antigens, herpes simplex viral antigens, varicella zoster viral antigens, Japanese encephalitis viral antigens, rabies viral antigens, cancer peptides selected from tumor associated antigens comprising antigens from leukemias and lymphomas, neurological tumors such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer, penile cancer, bone tumors, vascular tumors, cancer of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma, leukemia, carcinoembryonic antigen (CEA), prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67.

5. The composition of claim 1, wherein the ILT receptor antagonist comprises a mixture of an ILT2 receptor antagonist and an ILT4 receptor antagonist, wherein a ratio of the ILT2 receptor antagonist to the ILT4 receptor antagonist is 5:95, 10:90, 20:80, 25:75, 30:70, 40:60, 50:50, 60:40, 70:30, 75:25, 80:20, 90:10, and 5:95.

6. The composition of claim 1, wherein the composition is adapted for subcutaneous administration, intradermal administration, or both.

7. The composition of claim 1, wherein the ILT receptor antagonist is a small molecule receptor antagonist, a soluble protein, a fusion protein, an antibody or a fragment thereof, a polypeptide, a humanized antibody or antigenic-binding antibody fragments, or any combinations thereof.

8. A vaccine comprising one or more antigenic peptides and at least one antagonist that inhibits the binding of immunoglobulin-like transcript (ILT) receptor to CD8, wherein the vaccine is adapted for delivery to dermal CD14⁺ dendritic cells (DCs), wherein the antigenic peptides and the antagonist are provided in an amount effective to produce an immune response, a prophylaxis, a therapy or any combination thereof in a human or an animal subject.

9. The vaccine of claim 8, wherein the one or more antigenic peptides is further defined as a conjugate, wherein the conjugate comprises the antigenic peptide loaded, recombinantly linked or coupled chemically or with a recombinant linker to a dendritic cell (DC)-specific antibody or fragment thereof.

10. The vaccine of claim 8, further comprising one or more optional pharmaceutically acceptable carriers and adjuvants.

11. The vaccine of claim 8, wherein the antagonist is an ILT2, an ILT4, or an ILT5 antagonist.

12. The vaccine of claim 8, wherein the antagonist is humanized.

13. The vaccine of claim 8, wherein the antigenic peptides comprise at least one of a peptide or protein selected from gag, pol, env, Nef protein, reverse transcriptase, PSA-tetramer, a HIVgag-derived p24-PLA HIV gag p24 (gag), and other HIV components, hepatitis viral antigens, influenza viral antigens and peptides selected from the group consisting of hemagglutinin, neuraminidase, Influenza A Hemagglutinin HA-1 from a H1N1 Flu strain, HLA-A201-FluMP (58-66) peptide tetramer, and Avian Flu (HA5-1), measles viral antigens, rubella viral antigens, rotaviral antigens, cytomegaloviral antigens, respiratory syncytial viral antigens, herpes simplex viral antigens, varicella zoster viral antigens, Japanese encephalitis viral antigens, rabies viral antigens, cancer peptides selected from tumor associated antigens comprising antigens from leukemias and lymphomas, neurological tumors such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer, penile cancer, bone tumors, vascular tumors, cancer of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma, leukemia, carcinoembryonic antigen (CEA), prostate specific antigen (PSA), HER-2/neu, BAGE, GAGE, MAGE 1-4, 6 and 12, MUC (Mucin) (e.g., MUC-1, MUC-2), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67.

14. The vaccine of claim 8, wherein the ILT receptor antagonist comprises a mixture of an ILT2 receptor antagonist and an ILT4 receptor antagonist, wherein a ratio of the ILT2 receptor antagonist to the ILT4 receptor antagonist is 5:95, 10:90, 20:80, 25:75, 30:70, 40:60, 50:50, 60:40, 70:30, 75:25, 80:20, 90:10, and 5:95.

15. The vaccine of claim 8, wherein the vaccine is adapted for subcutaneous administration, intradermal administration, or both.

16. The vaccine of claim 8, wherein the ILT receptor antagonist is a small molecule receptor antagonist, a soluble ILT protein, a fusion protein, an antibody or a fragment thereof that binds specifically to an ILT protein, a polypeptide, or any combinations thereof.

17. A method for augmenting dendritic cell (DC) function, enhancing generation of polyfunctional CD8⁺ T cells by one or more dermal CD14⁺ dendritic cells (DCs), inducing generation of one or more cytotoxic T cells, or any combinations thereof in a human or animal subject comprising the steps of:
isolating and purifying an antigen-antibody conjugate comprising one or more dendritic cell (DC)-specific antibodies or fragments thereof and one or more native or engineered antigenic peptides;
providing at least one immunoglobulin-like transcript (ILT) receptor antagonist, wherein the ILT receptor is expressed on dermal CD14⁺ dendritic cells (DCs);
combining the antigen-antibody conjugate with the ILT receptor antagonist to form an immunostimulatory composition; and
introducing the composition into the human or animal subject to augment DC function, enhance generation of polyfunctional CD8⁺ T cells by one or more dermal CD14⁺ DCs, induce generation of one or more cytotoxic T cells, or any combinations thereof.

18. The method of claim 17, further comprising the optional step of measuring a level of one or more agents selected from the group consisting of IFN-γ, TNF-α, IL-2, IL-10, IL-4, IL-5, and IL-13, wherein a change in the level of the one or more agents is indicative of the augmented DC function, enhanced generation of polyfunctional CD8⁺ T cells by one or more dermal CD14⁺ DCs, increased generation of one or more cytotoxic T cells, or any combinations thereof.

19. The method of claim 17, wherein the antigenic peptides comprise at least one of a peptide or protein selected from gag, pol, env, Nef protein, reverse transcriptase, PSA-tetramer, a HIVgag-derived p24-PLA HIV gag p24 (gag), and other HIV components, hepatitis viral antigens, influenza viral antigens and peptides selected from the group consisting of hemagglutinin, neuraminidase, Influenza A Hemagglutinin HA-1 from a H1N1 Flu strain, HLA-A201-FluMP (58-66) peptide tetramer, and Avian Flu (HA5-1), measles viral antigens, rubella viral antigens, rotaviral antigens, cytomegaloviral antigens, respiratory syncytial viral antigens, herpes simplex viral antigens, varicella zoster viral antigens, Japanese encephalitis viral antigens, rabies viral antigens, cancer peptides selected from tumor associated antigens comprising antigens from leukemias and lymphomas, neurological tumors such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer, penile cancer, bone tumors, vascular tumors, cancer of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma and leukemia.

20. A method of providing immunostimulation by activation of one or more dendritic cells (DCs) to a human subject comprising the steps of:
identifying the human subject in need of immunostimulation for the prophylaxis, the therapy or a combination thereof against the one or more viral, bacterial, fungal, parasitic, protozoal, and parasitic diseases, and allergic disorders;
isolating one or more DCs from the human subject;
exposing the isolated DCs to activating amounts of a composition or a vaccine comprising:
one or more antigenic peptides, wherein the antigenic peptides are representative of one or more epitopes of the one or more antigens implicated or involved in the viral, the bacterial, the fungal, the parasitic, the protozoal, and the parasitic diseases, and the allergic disorders against which the immune response, the prophylaxis, the therapy, or any combination thereof is desired; and
at least one immunoglobulin-like transcript (ILT) receptor antagonist, wherein the ILT receptor is selected from the group consisting of ILT2, ILT4, ILT5, or any combinations thereof expressed on dermal CD14⁺ dendritic cells (DCs); and
reintroducing the activated DC complex into the human subject.

21. The method of claim 20, wherein the human subject is defined further as being a participant in a pre-clinical or a clinical trial.

22. The method of claim 20, further comprising the optional step of measuring a level of one or more agents selected from the group consisting of IFN-γ, TNF-α, IL-2, IL-10, IL-4, IL-5, and IL-13, wherein a change in the level of the one or more agents is indicative of the immunostimulation.

23. The method of claim 20, wherein the immunoglobulin-like transcript (ILT) receptor antagonist is humanized.

24. The method of claim 20, wherein the antigenic peptides comprise at least one of a peptide or protein selected from gag, pol, env, Nef protein, reverse transcriptase, PSA-tetramer, a HIVgag-derived p24-PLA HIV gag p24 (gag), and other HIV components, hepatitis viral antigens, influenza viral antigens and peptides selected from the group consisting of hemagglutinin, neuraminidase, Influenza A Hemagglutinin HA-1 from a H1N1 Flu strain, HLA-A201-FluMP (58-66) peptide tetramer, and Avian Flu (HA5-1), measles viral antigens, rubella viral antigens, rotaviral antigens, cytomegaloviral antigens, respiratory syncytial viral antigens, herpes simplex viral antigens, varicella zoster viral antigens, Japanese encephalitis viral antigens, rabies viral antigens, cancer peptides selected from tumor associated antigens comprising antigens from leukemias and lymphomas, neurological tumors such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer, penile cancer, bone tumors, vascular tumors, cancer of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma, leukemia, bacterial antigens selected from pertussis toxin, filamentous hemagglutinin, pertactin, FIM2, FIM3, adenylate cyclase and other pertussis bacterial antigen components, diptheria bacterial antigens, diptheria toxin or toxoid, other diptheria bacterial antigen components, tetanus bacterial antigens, tetanus toxin or toxoid, other tetanus bacterial antigen components, streptococcal bacterial antigens, gram-negative bacilli bacterial antigens, Mycobacterium tuberculosis bacterial antigens, mycolic acid, heat shock protein 65 (HSP65), Helicobacter pylori bacterial antigen components; pneumococcal bacterial antigens, haemophilus influenza bacterial antigens, anthrax bacterial antigens, and rickettsiae bacterial antigens, fungal antigens selected from candida fungal antigen components, histoplasma fungal antigens, cryptococcal fungal antigens, coccidiodes fungal antigens and tinea fungal antigens, protozoal and parasitic antigens antigens selected from plasmodium falciparum antigens, sporozoite surface antigens, circumsporozoite antigens, gametocyte/gamete surface antigens, blood-stage antigen pf 155/RESA, toxoplasma, schistosomae antigens, leishmania major and other leishmaniae antigens and trypanosoma cruzi antigens.

25. The method of claim 20, wherein the antigenic peptide comprises antigens involved in autoimmune diseases, allergy, and graft rejection selected from diabetes, diabetes mellitus, arthritis, multiple sclerosis, myasthenia gravis, systemic lupus erythematosis, autoimmune thyroiditis, dermatitis, psoriasis, Sjogren's Syndrome, alopecia greata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis.

26. The method of claim 20, wherein the antigenic peptides are selected from at least one of carcinoembryonic antigen (CEA), prostate specific antigen (PSA), HER-2/neu, BAGE, GAGE, MAGE 1-4, 6 and 12, MUC (Mucin) (e.g., MUC-1, MUC-2), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67.

27. The method of claim 20, wherein the antigenic peptide comprises antigens involved in allergic disorders selected from Japanese cedar pollen antigens, ragweed pollen antigens, rye grass pollen antigens, animal derived antigens, dust mite antigens, feline antigens, histocompatibility antigens, and penicillin and other therapeutic drugs.

28. A composition for modulating an immune response, suppressing an immune response, or both for a prophylaxis, a therapy or any combination thereof in a human or animal subject comprising:
one or more an anti-dendritic cell (DC)-specific antibodies, wherein the anti-DC-specific antibody may be a conjugate, wherein the conjugate comprises the one or more anti-DC-specific antibodies or fragments thereof loaded or chemically coupled with one or more antigenic peptides, wherein the antigenic peptides are representative of one or more epitopes of the one or more antigens implicated or involved in a disease or a condition against which the modulation or the suppression of the immune response for the prophylaxis, the therapy or any combination thereof is desired;
one or more immunoglobulin-like transcript (ILT) receptors, receptor agonist, receptor-like segments, or fragments thereof selected from the group consisting of ILT2, ILT4, ILT5, or any combinations thereof, wherein the ILT receptor is in a form of a fusion protein, a monomeric, dimeric or multimeric polypeptide complex, an antibody, or any combinations thereof; and
a pharmaceutically acceptable carrier, wherein the antibodies and the ILT receptors are each comprised in an amount such that, in combination with the other, are effective to modulate or suppress the immune response, for prophylaxis, for therapy or any combination thereof in the human or animal subject in need thereof.

29. The composition of claim 28, wherein the ILT antagonist is humanized.

30. The composition of claim 28, wherein the ILT receptors comprise ILT2, ILT4, or both.

31. The composition of claim 28, wherein the composition is used for the prophylaxis, the therapy, or both of one or more diseases or conditions selected from the group consisting of asthma, eczema, allograft rejection, graft-versus-host disease, hepatitis, and autoimmune disorders.

32. The composition of claim 28, wherein the one or more antigenic peptides comprise antigens involved in autoimmune diseases, wherein the autoimmune diseases are selected from the group consisting of diabetes, diabetes mellitus, arthritis, multiple sclerosis, myasthenia gravis, systemic lupus erythematosis, autoimmune thyroiditis, dermatitis, psoriasis, Sjogren's Syndrome, alopecia greata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis.

33. The composition of claim 28, wherein the composition stimulates secretion of one or more cytokines selected from the group consisting of IL-4, IL-5, IL-13, and IL-10.

34. The composition of claim 28, wherein the composition induces generation of one or more Type 2 cytokine-secreting CD8⁺ T cells (TC2).

35. A method for suppressing dendritic cell (DC) function, decreasing generation of polyfunctional CD8⁺ T cells by one or more dermal CD14⁺ dendritic cells (DCs), one or more cytotoxic T cells, or both, stimulating generation of one or more Type 2 cytokine-secreting CD8⁺ T cells (TC2), or any combinations thereof in a human or animal subject comprising the steps of:
isolating and purifying one or more dendritic cell (DC)-specific antibodies or a fragment thereof;
optionally loading or chemically coupling one or more native or engineered antigenic peptides to the DC-specific antibody to form an antibody-antigen conjugate;
providing one or more immunoglobulin-like transcript (ILT) receptors, receptor agonist, receptor-like segments, or fragments thereof selected from the group consisting of ILT2, ILT4, ILT5, or any combinations thereof obtained from one or more dermal CD14⁺ dendritic cells (DCs), wherein the ILT receptor is in a form of a fusion protein, a monomeric, dimeric or multimeric polypeptide complex, an antibody, or any combinations thereof;
contacting the antigen-antibody conjugate with the ILT receptor to form an immunosuppressive composition; and
introducing the composition into the human or animal subject to suppress dendritic cell (DC) function, decrease generation of polyfunctional CD8⁺ T cells by one or more dermal CD14⁺ dendritic cells (DCs), one or more cytotoxic T cells, or both, stimulate generation of one or more Type 2 cytokine-secreting CD8⁺ T cells (TC2), or any combinations thereof.

36. The method of claim 35, further comprising the optional step of measuring a level of one or more agents selected from the group consisting of IFN-γ, TNF-α, IL-2, IL-10, IL-4, IL-5, and IL-13, wherein a change in the level of the one or more agents is indicative of suppressed dendritic cell (DC) function, decreased generation of polyfunctional CD8⁺ T cells by one or more dermal CD14⁺ dendritic cells (DCs), one or more cytotoxic T cells, or both, stimulated generation of one or more Type 2 cytokine-secreting CD8⁺ T cells (TC2), or any combinations thereof.

37. The method of claim 35, wherein the method is used for treatment of one or more diseases or conditions in the human or animal subject requiring enhanced generation of TC2, asthma, eczema, allograft rejection, graft-versus-host disease, hepatitis, and autoimmune disorders, or any combinations thereof.

38. The method of claim 35, wherein the antagonists are humanized.
